(19) United States
(12) Patent Application Publication
(10) Pub. No.: US 2008/0113349 A1
(43) Pub. Date: May 15, 2008

(54) Method for detecting the presence of mammalian organisms using specific cytochrome c oxidase I (COI) and/or cytochrome b subsequences by a PCR based assay

(76) Inventors: Pranvera Ikonomi, Springfield, VA (US); Jason Cooper, Fairfax, VA (US); Greg Sykes, Fairfax, VA (US); Yvonne Reid, Haymarket, VA (US)

Correspondence Address: BIO INTELLECTUAL PROPERTY SERVICES (BIO IPS) LLC, 8509 KERNON CT., LORTON, VA, 22079, US

(21) Appl. No.: 11/592,117

(22) Filed: Nov. 3, 2006

(51) Int. Cl. C12Q 1/68; C07H 21/00; C12Q 1/00

(52) U.S. Cl. 435/6; 435/4; 536/24.33

(57) ABSTRACT

The present invention relates to a PCR (polymerase chain reaction) based assay that is useful for detecting, identifying, quantitating and analysis of a target nucleic acid (target nucleic acid hereinafter) in a sample. More specifically, the present invention relates to a PCR based assay that can improve accuracy in detecting, identifying, and quantitating contamination in mammalian cell lines using a PCR-based assay of nucleic acid oligonucleotides (oligoprobes) having a sequence expected to be complementary to a target nucleic acid sequence in a sample. More specifically, the sample may contain either cells or nucleic acid isolated from a cell line. The present invention also relates to a detection kit using the PCR-based assay. The invention also involves a method for using specifically produced nucleic acids complementary to specific sequences or populations of different sequences of the cytochrome c oxidase I (COI) and/or cytochrome b, to detect, identify, and quantify specific organisms, groups of organisms, groups of eukaryotic cells or viruses in cells.

### CROSS REFERENCE TO RELATED APPLICATIONS

Not Applicable.

### FEDERALLY SPONSORED RESEARCH

Not applicable

### SEQUENCE LISTING OR PROGRAM

Applicable.

### BACKGROUND OF INVENTION

### Field of Invention

The present invention relates to a PCR (polymerase chain reaction) based assay that is useful for detecting, identifying, quantitating and analysis of a target nucleic acid (target nucleic acid hereinafter) in a sample. More specifically, the present invention relates to a PCR based assay that can improve accuracy in detecting, identifying, and quantitating contamination in mammalian cell lines using a PCR-based assay of nucleic acid oligonucleotides (oligoprobes) having a sequence expected to be complementary to a target nucleic acid sequence in a sample. More specifically, the sample may contain either cells or nucleic acid isolated from a cell line. The present invention also relates to a detection kit using the PCR-based assay.

This invention also relates to genus-specific and species-specific oligonucleotides for identification of mammalian cell lines that are commonly used in a laboratory setting in a simple and rapid PCR based technique.

The invention also involves a method and means for detecting, identifying, and quantitating organisms in samples. Thus, it relates to a method for specifically and sensitively detecting and quantitating in a culture the presence of any contaminating mammalian organism containing the cytochrome c oxidase I (COI) and/or cytochrome b.

The invention also involves a method for using specifically produced nucleic acids complementary to specific sequences or populations of different sequences of the cytochrome c oxidase I (COI) and/or cytochrome b, to detect, identify, and quantify specific organisms, groups of organisms, groups of eukaryotic cells or viruses in cells.

This invention described and claimed herein also relates to probes and assays based on the use of genetic material such as DNA.

Our invention and the novelty, utility, and unobviousness thereof can be more clearly understood and appreciated when considered in light of the representative background information hereinafter set out.

### BACKGROUND OF THE INVENTION

The information provided below is not admitted to be prior art to the present invention, but is provided solely to assist the understanding of the reader. 0

A major problem with cell lines growing in cell cultures is the possibility of a cross-contamination with cells from unrelated cell lines. To avoid the negative impact of such contamination, continuous screening is essential for any cell culture. Most cell biologists recognize the need to perform routine testing for cell lines; however due to the cost and inaccuracies of the currently available tests, this has so far remained an unrealised ideal. Misidentification and cross-contamination of cell lines can also make scientific results and their reproducibility unreliable and represents a major problem of cell cultures. Cell lines are often maintained in culture for extended periods of time and are often exchanged between one laboratory and another. In exchanging cell lines there is often no guarantee of the real origin of the cell line as names are often truncated or misspelled with no good way to track the history of the material. Thus, many cell cultures used for research may not have been assayed for species identification for years. This is true also for material that might be used as a standard or control in a research protocol.

Historically the scientific community has been slow to recognize misidentification of cell lines as a major concern. Consequently, only nominal efforts have been made to test for and exclude cell lines that may have been cross contaminated with cell lines from another species where subsequent overgrowth has occurred (1). Researchers commonly give more attention to contamination by bacteria and fungi which are more readily detectable by changes in the morphology and behavior of the cells in culture (2). By contrast, cross contamination by other cell lines, like contamination by mycoplasma, is not readily detectable by these factors and can leave the appearance of the cells in culture unchanged. However, the awareness of the problems associated with mycoplasma contamination is continuously growing within the scientific community and several methods are made available for quick and rapid detection of such contaminations (4). Similarly, as the awareness of the effects of cell line cross-contamination becomes recognized, the need for such rapid and accurate detection platforms will become more acute. In addition to its use in detection of intraspecies contamination in research laboratories, the assay of the present invention could also be used in forensic analysis (5) and in the detection of contamination in food samples.

Cell culture overgrowth was first recognized as a problem in 1957 (1) and has since then been detected by a number of platforms including karyology, transplantation, and hemagglutination experiments (6, 7). In 1966, the isoenzyme polymorphism was implemented as a genetic marker and is now considered the standard method in species detection and detection of cell line cross-contamination and overgrowth and is used for quality control at major cell culture collections such as the ATCC (American Type Culture Collection (ATCC) P.O. Box 1549 Manassas, Va. 20108 USA). However, isoenzymology is costly, relatively slow and complex and not widely used in most laboratories maintaining cell cultures.

### Towards the Development of Rapid DNA-Based Diagnostic Tests

The availability of a rapid identification test would have a significant impact on the management of cell lines in cell cultures. For the identification of cells from unrelated cell lines in cell culture samples, DNA probe and DNA amplification technologies offer several advantages over conventional methods. There is no need for subculturing, hence the organism can be detected directly in cell culture samples thereby reducing the costs and time associated with isolation of organisms. DNA-based technologies have proven to be extremely useful for specific applications in the microbiology laboratory. The assay is particularly useful in detecting the presence in a mammalian cell culture containing a desired cell line of one or more contaminating cells from another undesirable mammalian cell line.

The present invention is an advantageous alternative to the conventional culture identification methods used in microbiology laboratories and in private clinics for routine diagnosis. Besides being much faster, DNA-based identification tests are more accurate than standard biochemical tests presently used for identification because the organism genotype (e.g. DNA level) is more stable than the bacterial phenotype (e.g. biochemical properties). The originality of this invention is that amplification primers or oligonucleotide probes are specific for 14 species of commonly encountered organisms. Amplification primers or oligonucleotide probes which are both derived from the sequence of species-specific DNA fragments are used as a basis to develop identification tests. It is clear to the individual skilled in the art that oligonucleotide sequences appropriate for the specific identification of the 14 organisms may be shorter or longer than the ones we have chosen and may be selected anywhere else in the identified species-specific sequences or selected data bank sequences. Alternatively, the oligonucleotides may be designed for use in amplification methods other than PCR.

Others have developed DNA-based tests for the detection and identification of some of the bacterial pathogens. However, their strategy was based on the amplification of the highly conserved 16S rRNA gene followed by hybridization with internal species-specific oligonucleotides. The assay of our invention is much simpler and more rapid because it allows the direct amplification of species-specific and genus-specific targets using the species-specific and genus-specific DNA fragments/oligonucleotides derived from mammalian organisms.

Multiplex PCR is a technique of PCR amplification which uses multiple pairs of primers to amplify multiple target sequences simultaneously in a single reaction tube. Use of multiplex PCR can significantly simplify experimental procedures in nucleic acid analysis and detection and shorten the time used. In addition, multiplex PCR requires no additional procedures and equipment. After first reported in 1988 (Chamberlain, J. S., et al., 1988. Nucleic Acids Res., 16, 11141-11156), multiplex PCR has become a fast and simple screening method for clinical and research laboratories. Multiplex PCR has been successfully used in areas including gene deletion analysis (Sieber, O. M., et al., 2002. Proc. Natl. Acad. Sci. U.S.A. 99, 2954-2958), gene mutation and gene polymorphism analysis (Moutou, C., et al., 2002. Eur. J. Hum. Genet. 10, 231-238), quantitative analysis of mRNA (Zimmermann, K., et al., 1996. Biotechniques 21, 480-484), RNA detection (Jin, L., et al., 1996. Mol. Cell Probes, 10,191-200; Zou, S., et al., 1998. J. Clin. Microbiol., 36, 1544-1548), and gene sequence analysis (Tettelin, H., et al., 1999. Genomics, 62, 500-507). In the area of diagnosis of infectious diseases, multiplex PCR already plays a significant role in identification and analytical research of virus (Druce, J., et al., 2002. J. Clin. Microbiol., 40, 1728-1732; Robert, P. Y., et al., 2002. J. Med. Virol., 66, 506-511), bacteria (Osek, J., 2002. Lett. Appl. Microbiol., 34, 304-310; Sloan, L. M., et al., 2002. J. Clin. Microbiol., 40, 96-100), parasite (Harris, E., et al., 1998. J. Clin. Microbiol., 36, 1989-1995), rapid determination of common mutations in glutathione S-transferase gene by PCR-based methods in healthy Chinese (Zhong et al. Clin Chim Acta. 2006 February; 364(1-2):205-8. Epub 2005 Aug. 10), for evaluation of the T-cell receptor V beta-chain repertoire (Fernandes et al., Clin Diagn Lab Immunol. 2005 April; 12(4):477-83), multiplex PCR detection of clinical and environmental strains of Vibrio vulnificus in shellfish (Panicker et al. Can J Microbiol. 2004 November; 50(11):911-22), rapid detection of common CARD15 variants in patients with inflammatory bowel disease (Roberts et al. Mol Diagn. 2004; 8(2):101-105), high-level multiplex DNA amplification (Broude et al. Antisense Nucleic Acid Drug Dev. 2001 October; 11(5):327-32), Real-time nucleic acid-based detection methods for pathogenic bacteria in food (McKillip et al. J Food Prot. 2004 April; 67(4):823-32), and drug tolerance of bacteria.

Very detailed primer design and multi-screening processes are usually required to establish an effective multiplex PCR. Problems often encountered in multiplex PCR are the imbalance of different target fragments amplified (some of the target fragments may not be effectively amplified at all) and relative low reproducibility. The following factors should be considered for a successful multiplex PCR program: the balance between concentrations of primers used, PCR buffer, Magnesium, and dNTP, temperature of each step in a PCR cycle, and the amount of template DNA and Taq DNA polymerase used. The optimization of denaturing temperature in PCR, the buffer system, the ratio of Magnesium and dNTP concentrations, and the concentration of primers are necessary for specificity in multiplex PCR. See Markoulatos, P., et al., 2002. Clin. Lab Anal., 16, 47-5 1; Elnifro, E. M., et al., 2000. Clin. Microbiol. Rev., 13, 559-570; and Henegariu, O., et al., 1997. Biotechniques, 23, 504-511. After studies of factors involved in multiplex PCR, a detailed optimizing program can be developed which requires multiple steps of optimization.

A common phenomenon in multiplex PCR is the amplification of one or more sequences, such that the ratio of PCR products is different (or even significantly different) from the ratio of the starting templates. This is caused by the limitation of polymerase and dNTP in the PCR system. The primers in the PCR system compete for the limited polymerase and dNTP, and the amplification efficiencies of these primers are different. Selection of primers, not the template, is important for the imbalance of multiplex PCR products (Suzuki, M. T. and Giovannoni, S. J. 1996. Appl. Environ. Microbiol., 62, 625-630). Thus, determination of the final concentration of each primer becomes a key factor for a multiplex PCR system. To avoid cross hybridization between primers and nonspecific template, each primer should be carefully designed and analyzed. For an ideal multiplex PCR, all primers should have the same amplification efficiency. The same efficiency of different primers can be established by designing similar Tm (melting temperature) for each primer (e.g., length of primer between 18 to 28 nucleotides, and GC content between 45 to 60%, with no homology between primers and no self homology). Optimizing concentration of each primer in multiplex PCR can be achieved by experiment.

As a result of development in methods for detecting and measuring genetic DNA on the basis of a hybridization process, rapid progress has been also been made in studies of gene chip (DNA chip, microarray) where a plurality of nucleic acid probes are immobilized on a substrate, allowing multiple detection tests on genetic DNA in a sample concomitantly. The method of detecting and measuring genetic DNA contained in a sample using a gene chip (DNA chip, microarray) is expected to be applied in various fields such as molecular biological research, gene disorder and infectious disease diagnosis.

The basic form of a microarray is a plurality of single-stranded nucleic acid fragments which are expected to have a sequence complementary to the sequence of a target gene or nucleic acid, where the single-stranded nucleic acid fragments are arranged in an array and immobilized on the surface of a substrate such as a glass substrate in order to detect target genetic DNA by the hybridization process. A single-stranded nucleic acid fragment to be used as a probe having a complementary base sequence to a target gene can be a chemically synthesized DNA oligomer called “oligonucleotide” or a complementary strand DNA fragment called “cDNA” which is enzymatically synthesized using a gene derived from a biomaterial as a template. As for immobilization of oligonucleotide-DNA on the surface of the substrate, methods are roughly classified into two catagories: one is to synthesize DNA molecules on the substrate sequentially after immobilizing one end to the substrate, the second is to synthesize the oligonucleotides and then to immobilize these nucleic acid fragments on a substrate using various bonding means.

As the means for immobilizing the separately prepared nucleic acid fragments on the substrate, various methods have been proposed, such as an adsorption-immobilizing method making use of the charge of a substrate and the charge of a nucleic acid fragment, and a coating-immobilizing method which is aimed to improve immobilization efficiency by using a film formed by coating the substrate surface with a poly-L-lysine or amino silane coupling agent.

Recently, the utility of microarrays comprised of gene-specific DNA or oligonucleotide probes was demonstrated to be a powerful tool for rapid detection and accurate speciation of a broad spectrum of different bacterial and viral species (Ivshina 2004, Volokhov, 2003, Volokhov 2004, Volokhov 2002). The results of these studies showed the combination of multiplexed PCR with microarray analysis of the amplified products as opposed to gel electrophoresis can significantly improve the reliability and throughput of detection. Therefore, microarray technology is a promising and potentially beneficial approach for the rapid and reliable detection of multiple organisms.

Two genes in particular make excellent potential targets for the development of a PCR based kit for species determination. They are both protein-coding mitochondrial genes. They provide an optimal target because these genes are conserved within a species while varying substantially between species. For example, every mouse cell line (mus musculus) must have virtually the same target sequence so that oligo primers can be designed that will pick up cells all such cell lines. Yet the sequence must vary enough so that common laboratory cell lines from, for example, rat (rattus norvegicus) and Chinese hamster (cricetulus griseus) are never amplified with the primer pair.

Mitochondrial genes encoding ribosomal (12S, 16S) DNA have often been used in the past to distinguish between cell types, but their utility is constrained by the prevalence of insertions and deletions (indels) that complicate sequence alignments. The 13 protein coding genes in the animal mitochondrial genome provide a better target, where indels are rare, since indels would to lead to a shift in the reading frame and therefore change the properties or the function of the product. COI is one of these protein coding genes and has been very well characterized. In addition many COI sequences are available in public databases and COI is the gene currently used by the Consortium for the Barcode of Life (CBOL), an international consortium housed at the Smithsonian Institute in Washington D.C., as the target gene in an ambitious initiative to DNA barcode all of animal life (http://barcoding.si.edu/), (8), (9)

CBOL has demonstrated that interspecific COI variation (for the first 648 base pairs of the gene) is quite substantial whereas intraspecific variation remains remarkably low. In one study for example, Hebert et. al. (2003) performed more than 13,000 pairwise comparisons based on COI sequences from 2,238 animal species (447 genera, 11 phyla) in GenBank (provided by the National Center For Biotechnology Information, National Institutes of Health, and National Library of Medicine). They found that about 80% of such pairs showed >8% sequence divergence, and that more than 98% of pairs exhibited >2% divergence. By contrast, individuals from the same species exhibited a level of sequence variation averaging less than 0.3%. This makes COI an ideal target for the design of species specific primer pairs. The COI and the cytochrome b genes have been previously reported as good targets for species identification (6), 10).

This invention provides specifically designed primer oligonucleotides for amplification of COI and cytochrome b that will provide identification of cells from 14 organisms for which cell line models are currently available and are used extensively in research laboratories. Moreover, the designed primer sets are used in multiplex-PCR assays and/or microarray assays for the detection and rapid identification of any cross-contaminating cells in a cell line or any DNA sample which may be used as a template.

We believe that the rapid and simple identification tests that we have developed will gradually replace the slower methods of conventional identification methods presently being used in microbiology laboratories and in private clinics.

### SUMMARY

The present invention relates to PCR based assay methods and materials for detecting and identifying contaminating cells from mammalian cell lines, as well as their related species and genera, that may be present as contaminates in a cell culture and can be detected in a test sample taken from such a cell culture.

In one embodiment, the invention is directed to a method using probes (DNA fragments and/or oligonucleotides) and/or amplification primers which are specific and sensitive for determining the presence and/or amount of nucleic acids from contamination cells of cell lines in any sample suspected of containing said nucleic acid from contaminating cells of cell lines, wherein said contaminating cells of cell lines nucleic acid or variant or part thereof comprise a selected target region hybridizable with said probes or primers; said method comprising the steps of contacting said sample with said probes or primers and detecting the presence and/or amount of hybridized probes and/or amplified products as an indication of the presence and/or amount of contaminating cells. In a preferred embodiment, the contaminating cells are from a mammalian cell line. In a more preferred embodiment, mammalian cell lines are selected from the group consisting of human, mouse, rat, cat, dog, bovine, pig, sheep, goat, horse, Chinese hamster, Green monkey, Rhesus monkey or rabbit.

In one embodiment, the method of the invention is performed directly on a sample obtained from a cell culture, environment or food. In another embodiment, the method is performed directly on a sample consisting of one or more cell lines. In another embodiment, cell line nucleic acid is amplified by a method selected from the group consisting of: a) polymerase chain reaction (PCR), b) ligase chain reaction, c) nucleic acid sequence-based amplification, d) self-sustained sequence replication, e) strand displacement amplification, f) branched DNA signal amplification, g) nested PCR, h) microarray, and I) multiplex PCR. In another embodiment, nucleic acid from cells of a contaminating cell line is amplified by PCR. In another embodiment the PCR protocol is modified to determine, within one hour, the presence of said the target nucleic acid present in a culture suspected of containing contaminating cells by performing for each amplification cycle an annealing step of only one second at 55° C. and a denaturation step of only one second at 95° C. without any elongation step.

In another embodiment, the invention is directed to a method for the detection, identification and/or quantification the presence of contaminating mammalian cells of cell lines directly from a test sample or from colonies taken from a culture or cell line suspected of being contaminated or subject to contamination, which comprises the following steps: a) depositing and fixing on an inert support or leaving in solution the contaminating mammalian cell line DNA of the sample, and lysing in situ said sample to release the DNA from the contaminating cells of mammalian cell lines, said contaminating mammalian cell lines DNA being in a substantially single stranded form; b) contacting said single stranded DNA with a probe, said probe comprising at least one single stranded nucleic acid which nucleotidic sequence is selected from the group consisting of SEQ ID NO:3, SEQ ID NO: 4, SEQ ID NO: 5, SEQ ID NO: 6, SEQ ID NO: 7 (or any other sequence from Table 1), a sequence complementary thereof, a part thereof and a variant thereof, which specifically anneals with contaminating mammalian cell line DNA or representatives of the DNA of contaminating mammalian cell lines, under conditions such that the nucleic acid of said probe can selectively hybridize with the DNA of the contaminating cells of mammalian cell lines, whereby a hybridization complex is formed, said complex being detected by labelling means, the label being present on said probe or the label being present on a first reactive member of said labelling means, said first reactive member reacting with a second reactive member present on said probe; and c) detecting the presence or the intensity of said label on said inert support or in said solution as an indication of the presence and/or amount of a specific contaminating mammalian cell lines in said test sample. In a preferred embodiment, the probe for detecting nucleic acid sequences from contaminating mammallian cell lines is selected from the group consisting of SEQ ID numbers that are listed in Table 1 and a sequence complementary thereof.

In another embodiment, the invention is directed to a method for detecting the presence and/or amount of contaminating cells, which may be present in a mammalian cell line or culture, by testing a sample, therefrom, wherein the test which comprises the following steps: a) treating said sample with an aqueous solution containing at least one pair of oligonucleotide primers having at least 12 nucleotides in length, one of said primers being capable of hybridizing selectively with one of the two complementary strands of the DNA isolated from or present in the contaminating cells of mammalian cell lines that contains a target sequence, and the other of said primers being capable of hybridizing with the other of said strands so as to form an extension product which contains the target sequence as a template, said at least one pair of primers being chosen from within one of the sequences that are listed in Table 1; b) synthesizing an extension product of each of said primers which extension products contain the target sequence, and amplifying said target sequence, if any, to a detectable level; and c) detecting the presence and/or amount of said amplified target sequence as an indication of the presence and/or amount of contaminating cells in mammalian cell lines in said test sample. In a preferred embodiment, the method uses at least one pair of primers selected from the sequences that are listed in Table 1. In a preferred embodiment, the method uses at least one pair of primers that is selected from the group consisting of: the sequences that are listed in Table 1.

In another embodiment, the invention is directed to a nucleic acid having the nucleotide sequence of any one of SEQ ID Nos that are listed in Table 1, a part thereof and variants thereof which, when in single stranded form, specifically hybridize with a target contaminating mammalian cell or cell line DNA as a probe or as a primer.

According to this invention, the oligonucleotides, primers or probes may be derived from either strand of the duplex DNA. The oligonucleotides, primers or probes may consist of the bases A, G, C, or T or analogs and they may be degenerated at one or more chosen nucleotide position(s). In one embodiment, the oligonucleotides, primers or probes may be of any suitable length and may be selected anywhere within the DNA sequences from proprietary fragments.

In another embodiment, the invention is directed to an oligonucleotide having a nucleotidic sequence of any one of SEQ ID Nos that are listed in Table 1.

In another embodiment, the invention is directed to a recombinant plasmid comprising a nucleic acid of any one of SEQ ID Nos that are listed in Table 1.

In another embodiment, the invention is directed to a recombinant host which has been transformed by a recombinant plasmid comprising a nucleic acid of any one of the SEQ ID Nos that are listed in Table 1. In a preferred embodiment, the recombinant host is Escherichia coli.

The various species-specific and genus-specific PCR assays which are embodiments of the present invention are all specific. For the PCR assays specific to species or genus, this means that DNA isolated from cells of a wide variety of species, other than that from the target species or genus and selected from Table 1, could not be amplified. For the PCR assay specific to cells from a human cell line, it means there was no amplification with genomic or subgenomic DNA from cells of other species (e.g., mouse, rat, cat, dog, bovine, pig, sheep, goat, horse, Chinese hamster, Green monkey, Rhesus monkey and rabbit).

The present invention relates to assay methods and materials for detecting and identifying different cells or nucleic acid molecules from species such as human, mouse, rat, cat, dog, bovine, pig, sheep, goat, horse, Chinese hamster, Green monkey, Rhesus monkey and rabbit that contaminate a test sample, such as a sample from a cell culture.

The present invention provides a method for detecting or identifying different cells or nucleic acid molecules from species such as human, mouse, rat, cat, dog, bovine, pig, sheep, goat, horse, Chinese hamster, Green monkey, Rhesus monkey and rabbit, present as a contaminant in a cell culture, comprising the following steps: a) if necessary, extracting nucleic acids from a sample such as cell lines, biological products or human tissue or serum; b) amplifying target DNA of different species such as human, mouse, rat, cat, dog, bovine, pig, sheep, goat, horse, Chinese hamster, Green monkey, Rhesus monkey and rabbit using more than one proper primer and c) detecting signals generated from the hybridization reaction.

From the detected signals in the step c), the existence of different cells or nucleic acid from species such as human, mouse, rat, cat, dog, bovine, pig, sheep, goat, horse, Chinese hamster, Green monkey, Rhesus monkey and rabbit and its related strains in the sample can be determined.

The present inventors carried out a sequence analysis of the cytochrome c oxidase I (COI) and/or cytochrome b to obtain species-specific and genus-specific oligonucleotides for detecting the presence of cells from various mammals and mammals-related species which can be a basis of developing a specific and sensitive hybridization assay. In one embodiment, the present invention provides nucleic acids that can be used in detecting the presence of cells from cell lines of various mammal species and genera. The nucleic acids can be oligonucleotides that can function as primers for PCR reactions. The nucleic acids can also be primers or genomic or sub-genomic nucleic acids that can be used as controls for monitoring the progression, specificity, and/or sensitivity of the methods of the invention.

In one embodiment, oligonucleotides according to the invention can specifically hybridize to nucleic acids of human, mouse, rat, cat, dog, bovine, pig, sheep, goat, horse, Chinese hamster, Green monkey, Rhesus monkey and rabbit. Suitable primers can be designed by those of skill in the art based on known nucleic acid sequences of this invention to identify the cells or nucleic acid of human, mouse, rat, cat, dog, bovine, pig, sheep, goat, horse, Chinese hamster, Green monkey, Rhesus monkey and rabbit in a cell culture.

It is another embodiment of the present invention to provide a microarray comprising genus-specific and species-specific oligonucleotides for detecting cells of human, mouse, rat, cat, dog, bovine, pig, sheep, goat, horse, Chinese hamster, Green monkey, Rhesus monkey and rabbit in a cell culture and its related species and genera as oligonucleotides.

It is another embodiment of the present invention to provide a method for detecting cells of human, mouse, rat, cat, dog, bovine, pig, sheep, goat, horse, Chinese hamster, Green monkey, Rhesus monkey and rabbit in a cell culture and its related species and strains using a PCR based assay such as multiplexed PCR and microarray.

It is another embodiment of the present invention to provide methods (i.e., assays) that detect more than one species of a given genus. That is, the present invention contemplates detecting the cells or nucleic acid of multiple members of the cells of human, mouse, rat, cat, dog, bovine, pig, sheep, goat, horse, Chinese hamster, Green monkey, Rhesus monkey and rabbit in the same assay. Therefore, the present invention targets a genomic sequence that is well conserved among the human, mouse, rat, cat, dog, bovine, pig, sheep, goat, horse, Chinese hamster, Green monkey, Rhesus monkey and rabbit.

For the purposes of the present invention, oligonucleotides of human, mouse, rat, cat, dog, bovine, pig, sheep, goat, horse, Chinese hamster, Green monkey, Rhesus monkey and/or rabbit, were identified wherein attention was primarily focused on cytochrome c oxidase I (COI) and/or cytochrome b subsequences. Oligonucleotides synthesis can be carried out by any known method. For example, oligonucleotides can be produced using cyanoethyl phosphoramidite chemistry, ammonium hydroxide deprotection, and desalting by gel filtration chromatography. If desired, primers can be further purified by high performance liquid chromatography (HPLC), polyacrylamide gel electrophoresis (PAGE), or any other method known to those of skill in the art.

Nucleic acids other than oligonucleotides are also part of this invention. These nucleic acids can be used as controls for monitoring various aspects of the methods of the present invention. The control nucleic acids can be cytochrome c oxidase I (COI) and/or cytochrome b nucleic acids, which can be used as positive controls to confirm that the methods and oligonucleotides are suitable for amplification and detection of cytochrome c oxidase I (COI) and/or cytochrome b subsequences nucleic acids. The control nucleic acids can also be other mammalian nucleic acids or eukaryotic nucleic acids. These other nucleic acids can be used to confirm the specificity of the primers used to amplify and detect cytochrome c oxidase I (COI) and/or cytochrome b nucleic acids and to confirm that no inhibitors of amplification were present in the amplification mixtures. Control nucleic acids can be genomic or sub-genomic nucleic acids.

In the detection method according to the present invention, the sample may be a biological drug, cell line, or human tissues, mammalian tissues, or serum.

It is another embodiment of the present invention to provide a kit for identifying cells from cell lines of human, mouse, rat, cat, dog, bovine, pig, sheep, goat, horse, Chinese hamster, Green monkey, Rhesus monkey and rabbit as well as its related species and genera individually or simultaneously, comprising genus-specific and species-specific oligonucleotides for cells of human, mouse, rat, cat, dog, bovine, pig, sheep, goat, horse, Chinese hamster, Green monkey, Rhesus monkey and rabbit and its related species and genera.

The cytochrome c oxidase I (COI) and/or cytochrome b subsequence of the target DNA of the present invention can be used indirectly for designing probes or primers used for identifying cells and/or nucleic acid from human, mouse, rat, cat, dog, bovine, pig, sheep, goat, horse, Chinese hamster, Green monkey, Rhesus monkey and rabbit species as well as related species and genera, or directly for identifying cells and/or nucleic acid from human, mouse, rat, cat, dog, bovine, pig, sheep, goat, horse, Chinese hamster, Green monkey, Rhesus monkey and rabbit species as well as related species and genera via PCR amplification.

According to another aspect of the present invention, there is provided an oligonucleotide for genus-specific, species-specific or human, mouse, rat, cat, dog, bovine, pig, sheep, goat, horse, Chinese hamster, Green monkey, Rhesus monkey and rabbit, comprising a sequence selected from SEQ ID Nos. as listed in Table (1) or its complementary sequence.

According to one embodiment of the invention, the oligonucleotides can be used as primers for PCR amplification in order to identify cells or nucleic acid from human, mouse, rat, cat, dog, bovine, pig, sheep, goat, horse, Chinese hamster, Green monkey, Rhesus monkey and rabbit cell lines as well as their related species or genera or as probes for hybridization reaction in order to identify human, mouse, rat, cat, dog, bovine, pig, sheep, goat, horse, Chinese hamster, Green monkey, Rhesus monkey and rabbit cell lines as well as their related species and genera.

The purifying step of the nucleic acid can be performed using a conventional DNA or RNA purification method or kit. According to another aspect of the present invention, there is provided a kit for identification of contaminated cell lines including, but not limited to, contaminated mammalian cell lines, comprising more than one oligonucleotide selected from species-specific and/or genus-specific oligonucleotides for identifying contaminating cells from, but not limited to, mammalian cell lines such as human, mouse, rat, cat, dog, bovine, pig, sheep, goat, horse, Chinese hamster, Green monkey, Rhesus monkey and rabbit according to the above present invention.

The present invention provides kits for detecting the contamination of cell lines by cells from various mammalian genera and species which may be present in a sample. In its most basic form, the kit of the invention can comprise one or more nucleic acids or compositions containing such nucleic acids provided by the present invention. The kits can comprise the components in a single package or in more than one package within the same kit. Where more than one package is included within a kit, each package can independently contain a single component or multiple components, in any suitable combination. As used herein, a combination of two or more packages or containers in a single kit is referred to as “in packaged combination”. The kits and containers within the kits can be fabricated with any known material. For example, the kits themselves can be made of a plastic material or cardboard. The containers that hold the components can be, for example, a plastic material or glass. Different containers within one kit can be made of different materials. In embodiments, the kit can contain another kit within it. For example, the kit of the invention can comprise a kit for purifying nucleic acids.

In general, the kits can comprise, in a single package or in packaged combination, two or more oligonucleotides (primers), reagents and/or other components for performing the methods of the invention, nucleic acid templates for use as positive controls or specificity controls, or combinations of two or more of these.

In similar embodiments, the kit according to the invention comprises at least two primers, where each of the primers has a sequence comprising a SEQ ID NO (the sequence can be selected from sequences that are encompassed by this invention), where each primer present in the kit has a sequence that differs from each other primer sequence in the kit. In these embodiments, the kit comprises two primers, one comprising the sequence of SEQ ID NO:1 and the other comprising the sequence of SEQ ID NO:2 (for example—the sequence can be selected from sequences of this invention). In other embodiments, the kit comprises three primers, one comprising the sequence of SEQ ID NO:1, the second comprising the sequence of SEQ ID NO:2, and the third primer comprising the sequence of SEQ ID NO:3, SEQ ID NO:4, or SEQ ID NO:5 (for example). In an exemplary embodiment, the kit comprises a primer comprising the sequence of SEQ ID NO:1, a primer comprising the sequence of SEQ ID NO:2, a primer comprising the sequence of SEQ ID NO:3, and a primer comprising the sequence of SEQ ID NO:4. In yet another exemplary embodiment, the kit comprises a primer comprising the sequence of SEQ ID NO:1, a primer comprising the sequence of SEQ ID NO:2, a primer comprising the sequence of SEQ ID NO:3, a primer comprising the sequence of SEQ ID NO:4, and a primer comprising the sequence of SEQ ID NO:5. Other primer sequences of this invention may be used as part of this invention. In certain embodiments, all of the primers provided in the kit are provided in a single container, whereas in other embodiments, they are provided in at least two separate containers, alone or in combination with one or more other primer. In a specific embodiment, the kit can comprise a set of two or more, and preferably four, five or any of those primers that are encompassed by this invention as described herein, that ecognize and amplify specific cytochrome c oxidase I (COI) and/or cytochrome b subsequences. In the present invention, the probes may be a combination of more than one probe capable of simultaneously detecting many contaminations of the tested cell lines, including but not limited to, contaminating mammalian cell lines and their related mammalian genera and mammalian species from a single sample. Practically, the probes are optimized to simultaneously hybridize with multiple target DNAs of cell lines of mammalian and as well as cell lines of mammalian related genera and species under the same hybridization and washing conditions.

Accordingly, the invention provides a kit comprising at least one container holding a composition comprising at least one oligonucleotide primer, each of these primers having a sequence comprising the sequence of one or more of the sequences according to the invention. For example, the kit can comprise a container containing a primer having a sequence comprising SEQ ID NO:1, and a container containing a primer having a sequence comprising SEQ ID NO:2. Likewise, other containers can be provided that contain a primer having a sequence comprising SEQ ID NO:3, a primer having a sequence comprising SEQ ID NO:4, and a primer having a sequence comprising SEQ ID NO:5 and so on. Alternatively, two or more primers can be contained in one container, the invention not being limited by any particular combination of primers in each container. In one aspect of the invention, containers can be provided that contain primers having all of the sequences that are listed in the invention.

The kit of the invention can comprise primers for the amplification control (AC). The AC primers may be contained in a container separate from the other components of the kit. In another embodiment, the AC primers are contained in the same container as the mammalian primers. In another possible embodiment, the AC primers are contained in the same container as the AC template nucleic acid. Similarly, the AC primers, the AC template nucleic acid, and at least one mammalian primer may be contained in the same container. In one embodiment, the mammalian primer is selected from the group of human, mouse, rat, cat, dog, bovine, pig, sheep, goat, horse, Chinese hamster, Green monkey, Rhesus monkey and rabbit primers according to the above present invention. In exemplary embodiments, the AC primers individually comprise the sequences of one or more the sequences of this invention.

The kit of the invention can comprise purified mammalian nucleic acids, including but not limited to, purified nucleic acid that is isolated from human, mouse, rat, cat, dog, bovine, pig, sheep, goat, horse, Chinese hamster, Green monkey, Rhesus monkey and rabbit according to the above present invention.

These nucleic acids of the present invention can be genomic or sub-genomic nucleic acids. In exemplary embodiments, the kits comprise a container containing purified gDNA of mammals, including but not limited to, purified genomic (gDNA) that is isolated from human, mouse, rat, cat, dog, bovine, pig, sheep, goat, horse, Chinese hamster, Green monkey, Rhesus monkey and rabbit.

In other embodiments, a known amount of mammal or mammal related genera and species gDNA is contained in a single container within the kit. In certain cases, two containers, each containing one or the other genomic DNA (gDNA) of mammals, related species and genera, are included in the kit. In certain other cases, a human gDNA and a mouse gDNA are included in a single container within the kit. In other embodiments, gDNA from each mammalian related species and genera is provided in the kit, either in separate containers or together in a single container.

The kit of the invention can comprise one or more components useful for amplifying target sequences of mammals, including but not limited to, target sequences of human, mouse, rat, cat, dog, bovine, pig, sheep, goat, horse, Chinese hamster, Green monkey, Rhesus monkey and rabbit according to the above present invention.

In other embodiments, some or all of the reagents and supplies necessary for performing PCR are included in the kit, non-limiting examples of reagents are buffers (e.g., a buffer containing Tris®, HEPES® and the like), salts, and a template-dependent nucleic acid extending enzyme (such as a thermostable enzyme, such as Taq polymerase), a buffer suitable for activity of the enzyme, and additional reagents needed by the enzyme, such as dNTPs, dUTP, and/or a UDG enzyme. In other embodiments, the kit comprises Brilliant® SYBR® Green QPCR Master Mix (Catalog #600548, Stratagene, La Jolla, Calif.). A non-limiting example of supplies is reaction vessels (e.g., microcentrifuge tubes).

The kit can comprise at least one dye for detecting nucleic acids, including, but not limited to, dsDNA. The kit may comprise a sequence-non-specific dye that detects dsDNA, such as SYBR® Green dye (Molecular Probes, Eugene, Oreg.). The dye is preferably contained alone in a separate container. In other embodiments, the dye is provided as a concentrated stock solution, for example, as a 50× solution. In other embodiments, the kit comprises a passive reference dye. The passive reference dye can be included in the kit alone in a separate container. The passive reference dye can be provided as a concentrated stock solution, for example, as a 1 mM stock solution. A non-exclusive exemplary passive reference dye is ROX dye. In other embodiments, the kit contains either a DNA-detecting dye or a passive reference dye. In other embodiments, the kit contains both a DNA-detecting dye and a passive reference dye.

The kit can also comprise one or more components useful for purifying nucleic acids. These components are particularly suited for purifying nucleic acid isolated from mammals, including but not limited to, nucleic acid that is isolated from human, mouse, rat, cat, dog, bovine, pig, sheep, goat, horse, Chinese hamster, Green monkey, Rhesus monkey and rabbit cell lines according to the above present invention.

The components can be, among other things, reagents and supplies that can be used to purify nucleic acids. Non-limiting examples of such reagents and supplies include a DNA binding solution, a wash buffer, and containers, such as microcentrifuge tubes, for collection of binding solutions, wash buffers, and purified nucleic acids. The components can also contain a resin, gel, or other substance that is useful for purifying nucleic acids. In one embodiment, the kit comprises the components of the StrataPrep® PCR Purification Kit (Catalog #400771, Stratagene, La Jolla, Calif.).

In one embodiment, this invention provides a microarray comprising more than one primer selected from genus-specific or species-specific primers for identifying cells of human, mouse, rat, cat, dog, bovine, pig, sheep, goat, horse, Chinese hamster, Green monkey, Rhesus monkey and rabbit wherein the primer(s) is attached to a support.

The microarray according to the present invention can be used for simultaneously identifying various cell lines of human, mouse, rat, cat, dog, bovine, pig, sheep, goat, horse, Chinese hamster, Green monkey, Rhesus monkey and rabbit, as well as their related species and genera which are known as a contaminant of biological drug or cell lines. The microarray may, also, comprise human, mouse, rat, cat, dog, bovine, pig, sheep, goat, horse, Chinese hamster, Green monkey, Rhesus monkey and rabbit-specific oligonucleotides for identifying human, mouse, rat, cat, dog, bovine, pig, sheep, goat, horse, Chinese hamster, Green monkey, Rhesus monkey and rabbit cell line and its related genera and species as a set attached on a support.

The present invention provides a microarray comprising a set of probes which can simultaneously detect a cell of human, mouse, rat, cat, dog, bovine, pig, sheep, goat, horse, Chinese hamster, Green monkey, Rhesus monkey and rabbit and its related species and genera from a single sample with a single experiment.

According to another embodiment of the present invention, there is provided a method for detecting a cell line of human, mouse, rat, cat, dog, bovine, pig, sheep, goat, horse, Chinese hamster, Green monkey, Rhesus monkey and rabbit, comprising the following steps: a) extracting nucleic acids from a sample; b) amplifying target DNA among the extracted nucleic acids; c) hybridizing the amplified target DNA with probes of the microarray or multiplexed PCR according to the present invention; and d) detecting signals generated from the hybridization reaction.

In one embodiment, this invention provides a microarray comprising more than one oligonucleotide selected from cytochrome c oxidase I (COI) or cytochrome b oligonucleotides for detecting and identifying contaminating cells from various cell lines of mammalian related species and genera.

In one embodiment of this invention, the probes are selected from a group consisting of DNA, RNA, PNA, LNA and HNA.

In one embodiment of this invention, the support is selected from a group consisting of slide glass, plastic, membrane, semiconductive chip, silicon and gel.

In one embodiment, this invention provides a method for detecting the presence of contaminating cells from a mammalian cell line which may improperly be present in another cell line, including another mammalian cell line, comprising the following steps: a) extracting nucleic acids from a sample; b) amplifying target DNA among the extracted nucleic acids; c) hybridizing the amplified target DNA with probes of the microarray according to one or more of the embodiments of this invention; and d) detecting signals generated from the hybridization reaction.

In one embodiment, this invention provides a composition comprising at least one oligonucleotide primer, each of said primer having a sequence comprising any one sequence selected from SEQ ID Nos. ______ as shown in TABLE 1.

In one embodiment, this invention provides a composition, further, comprising a primer having a sequence comprising a sequence selected from the group consisting of sequences that are listed in TABLE 1. In a preferred embodiment, the composition comprises an amplification control nucleic acid and at least one oligonucleotide primer specific for said amplification control nucleic acid. In a preferred embodiment, the composition is further comprising Taq polymerase. In a preferred embodiment, the composition is comprising a dye that can specifically detect double stranded DNA. In a preferred embodiment, the kit, further, comprises genomic or sub-genomic mammalian cell line nucleic acids in a second container. In a preferred embodiment, the kit further comprises an amplification control nucleic acid, wherein said amplification control nucleic acid is present in said first container or in a second container.

In a preferred embodiment, the kit further comprises reagents and supplies for purification of nucleic acids. In a preferred embodiment, the kit further comprises, in packaged combination, SYBR Green dye, an amplification control, a mammalian control, a reference dye, a template dependent nucleic acid extending enzyme, and all reagents and supplies necessary to purify nucleic acids.

In one embodiment, this invention further comprises providing an amplification control nucleic acid and at least two primers that specifically hybridize to the amplification control; amplifying the amplification control; and detecting the product of the amplification control amplifying reaction. In one embodiment, amplifying and detecting of the nucleic acid from a contaminating mammalian cell or cell line and amplifying and detecting of amplification control are performed in a different reaction tube. In one embodiment, this invention comprises amplifying and detecting of purified nucleic acid isolated from contaminating mammalian cells or a cell line and amplifying and detecting of amplification control in the same reaction tube.

In one embodiment, this invention is directed to a hybridization platform comprising more than one oligonucleotide selected from cytochrome c oxidase I (COI) or cytochrome b oligonucleotides for detecting and identifying cells from a contaminating mammalian cell line using probes that are attached on a support. In a preferred embodiment, the probes are any one selected from a group consisting of DNA, RNA, PNA, LNA and HNA. In a preferred embodiment, the support is any one selected from a group consisting of slide glass, plastic, membrane, semiconductive chip, silicon and gel.

In one embodiment, this invention is directed to a method for detecting contaminating cells from a mammalian cell line, comprising the following steps: a) extracting nucleic acids from a sample; b) amplifying target DNA among the extracted nucleic acids; c) hybridizing the amplified target DNA with probes of the hybridization platform according to embodiments of this invention; and d) detecting signals generated from the hybridization reaction. In preferred embodiment, the hybridization platform is selected from the group consisting of PCR, FLAP endonuclease assay, TAQMAN quantitative PCR, SCORPION PCR, multiplex PCR, and microarrays.

In another embodiment, this invention comprises a method of detecting the presence of contaminating mammalian cells from a cell line in a sample, the method comprising: (a) isolating nucleic acid from the sample, (b) performing a multiplex polymerase chain reaction, wherein a plurality of nucleic acid fragments representing a plurality of mammalian cell lines are amplified by a plurality of forward and reverse primers; and (c) determining that a sample is positive for the presence of the contaminating mammalian cells or cell line if a number of identified and amplified nucleic acid fragments are sufficient to suggest the presence of the contaminating mammalian cells or cell line in the sample.

In another embodiment, the contaminating cells of a cell line are selected from the group consisting of human, mouse, rat, cat, dog, bovine, pig, sheep, goat, horse, Chinese hamster, Green monkey, Rhesus monkey and rabbit

In another embodiment, the plurality of primers is selected from the group consisting of primers whose DNA sequences listed in Table 1. In another embodiment, the primers comprise the following characteristics:

(a) about twenty to thirty bases long;
(b) melting temperature of about 60° C.;
(c) GC content of about fifty percent;
(d) minimal dimer formation; and
(e) low frequency of mutations in the primer binding site.

In another embodiment of the invention, the primers are isolated from specific cytochrome c oxidase I

### (COI) and/or cytochrome b subsequences.

In another embodiment, this invention is directed to a method for detecting cross contamination in a sample, the method comprising:

(a) performing a multiplex polymerase chain reaction, wherein a plurality of primers amplify a plurality of mammalian nucleic acid fragments which are selected from the group consisting of nucleic acid molecules of human, mouse, rat, cat, dog, bovine, pig, sheep, goat, horse, Chinese hamster, Green monkey, Rhesus monkey and rabbit.
(b) determining that a sample is positive for human, mouse, rat, cat, dog, bovine, pig, sheep, goat, horse, Chinese hamster, Green monkey, Rhesus monkey or rabbit if a number of amplified fragments is sufficient to detect the human, mouse, rat, cat, dog, bovine, pig, sheep, goat, horse, Chinese hamster, Green monkey, Rhesus monkey or rabbit in the sample.

In a preferred embodiment, the multiplex polymerase chain reaction is performed in a single reaction chamber. In a more preferred embodiment, the plurality of mammalian nucleic acid fragments is selected from the group consisting of RNA, cDNA, and genomic DNA; wherein the mammalian nucleic acid fragments may be isolated from human, mouse, rat, cat, dog, bovine, pig, sheep, goat, horse, Chinese hamster, Green monkey, Rhesus monkey, rabbit, or related genera and species according to the above present invention. In another embodiment, this invention is directed to the multiplex polymerase chain reaction which is performed with an isolated nucleic acid.

In a more preferred embodiment, the multiplex polymerase chain reaction is performed directly with a biological sample selected from the group consisting of a mammalian cell culture or a mix of mammalian and bacterial culture.

In another preferred embodiment, the DNA is isolated from a clinical sample. In another preferred embodiment, the primer pairs are selected from the primers listed in TABLE 1.

In another embodiment, this invention is directed to a method of detecting contaminating mammalian cell lines in a sample, the method comprising: (a) designing a plurality of primers [from] in a plurality of subsequences of cytochrome c oxidase I (COI) or cytochrome b, wherein the primers are (i) specific for cytochrome c oxidase I (COI) or cytochrome b subsequences; (ii) have a low frequency of mutations in primer binding sites; (b) performing a multiplex polymerase chain reaction wherein the plurality of primers amplify a plurality of nucleic acid fragments representing the plurality of subsequences of cytochrome c oxidase I (COI) or cytochrome b; and (c) determining that a sample is positive for a particular contamination from a mammalian cell line. In one embodiment, the contaminating cell line is selected from the group consisting of human, mouse, rat, cat, dog, bovine, pig, sheep, goat, horse, Chinese hamster, Green monkey, Rhesus monkey and rabbit.

In another embodiment, this invention is directed to a diagnostic kit useful to identify contaminating mammalian cells or cell line in a sample comprising: (a) a plurality of primers to amplify a plurality of nucleic acid fragments representing a plurality of cytochrome c oxidase I (COI) and/or cytochrome b subsequences; and (b) reagents to perform a multiplex polymerase chain reaction.

In a preferred embodiment, the reagents comprise a DNA polymerase, nucleotides, and buffers.

In a more preferred embodiment, the primers comprise a plurality of DNA molecules comprising nucleotide sequences listed in TABLE 1.

In one embodiment, the invention is directed to a nucleic acid having the nucleotide sequence of any one of SEQ ID Nos that are listed in TABLE 1, a part thereof and variants thereof which, when in single stranded form, specifically hybridize with a target nucleic acid from a contaminating mammalian cell or cell lines DNA as a probe or as a primer.

In one embodiment, the invention is directed to an oligonucleotide having a nucleotidic sequence of any one of SEQ ID Nos that are listed in TABLE 1.

In one embodiment, the invention is directed to a recombinant plasmid comprising a nucleic acid any one of SEQ ID Nos that are listed in TABLE 1.

In one embodiment, the invention is directed to a recombinant host which has been transformed by a recombinant plasmid comprising a nucleic acid of any one of SEQ ID Nos that are listed in TABLE 1. In a preferred embodiment, the recombinant host is Escherichia coli.

In one embodiment, the invention is directed to diagnostic kits for the detection and/or quantification of the nucleic acids of any combination of the contaminating mammalian cells or cell lines described herein comprising any combination of probes defined herein.

In one embodiment, the invention is directed to diagnostic kits for the detection and/or quantification of the nucleic acids of any combination of the contaminating mammalian cells or cell lines described herein, comprising any combination of oligonucleotide probes defined herein.

In one embodiment, the invention is directed to diagnostic kits for the detection and/or quantification of the nucleic acids of any combination of the contaminating mammalian cells or cell lines described herein comprising any combination of primers defined herein.

In one embodiment, the invention is directed to diagnostic kits for the simultaneous detection of nucleic acids of any combination of the contaminating mammalian cells or cell lines described herein comprising any combination of the probes defined herein.

In one embodiment of this invention, the probes may be any materials having base sequence, preferably any one selected from a group consisting of DNA (Deoxyribose Nucleic acid), RNA (Ribose Nucleic Acid), and nucleic acid analogues such as PNA (Peptide Nucleic Acid), LNA (Locked Nucleic Acid) and HNA (Hexitol Nucleic Acid).

In one embodiment of this invention, the support may be any materials to which the probes can be attached, preferably one selected from a group consisting of slide glass, plastic, membrane, semiconductive chip, silicon and gel. The microarray according to the present invention can be manufactured using conventional methods such as pin microarray, ink jet, photolithography or electric array method.

### BRIEF DESCRIPTION OF THE FIGURES

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown. Drawings are not necessary to scale. Certain features of the invention may be exaggerated in scale or shown in schematic form in the interest of clarity and conciseness.

FIG. 1. Size range of species specific PCR Fragments. Oligonucleotide sets for fourteen animal and human targets have been designed to produce amplified product that increases in a step-wise fashion for each species. This stepwise design is essential for the development of a multiplexed assay that can be resolved by simple agarose gel electrophoresis. In the figure, each blue square represents the size of the amplified product for a different species. The range is extended from 94 bp to 460 pb and no amplified product is within 20 bp of any other. Thus the size of the amplified product becomes a signature marker for presence of a particular species in a cell culture mix.

FIG. 2. PCR amplification with species-specific oligonucleotide pairs. Amplified products were detected by ethidium bromide staining after agarose gel electrophoresis in a 4% pre-casted gel. The exact size of each product is indicated by the marker column (M1) and is shown in blue. Each lane across the gel shows amplified product specific for a different species for the 14 species in the assay: Lane 1, bovine specific product (94 bp); Lane 2, goat (115 bp); Lane 3, rabbit (133 bp), Lane 4, mouse (150 bp); lane 5, dog (172 bp); Lane 6, rat (196 bp); Lane 7, African green monkey (222 bp); Lane 8, Horse (243 bp); Lane 9, sheep (265 bp); Lane 10, Rhesus monkey (287 bp), Lane 11 Chinese hamster (315 bp); Lane 12 Chinese hamster repeat for reference on the second gel fragment; Lane 13, cat (341 bp); Lane 14, human (391 bp); Lane 15, pig (460 bp); (M2), second marker showing sizes for the second gel fragment. As can be seen the amplified products increase in size from bovine (94 bp) to pig (460 bp) in an easily resolvable step-wise fashion.

FIG. 3. Identification of 14 species by “speciation kit” with internal control. Amplified fragments were detected by ethidium bromide staining on a 2% agarose gel. The label on the far left hand size shows the lengths of the fragments. The internal control can be seen as a band in all PCR reactions at 70 bp and has been multiplexed with each particular primer pair. Note that a few of the primers have been modified slightly from FIG. 2. Lane M, the 100 bp marker (Invitrogen) is present on either side of the gel. Lane 1, pig specific product (460 bp); Lane 2, human (391 bp); Lane 3, cat (341 bp), Lane 4, chinese hamster (315 bp); Lane 5, Rhesus monkey (287 bp); Lane 6, sheep (267 bp); Lane 7, horse (243 bp), Lane 8, African green monkey (222 bp), Lane 9, rat (196 bp), lane 10, dog (172 bp), Lane 11 mouse (150 bp); Lane 12, rabbit (136 bp); Lane 13, goat (117 bp); Lane 14, bovine (102 bp); Lane 15, internal control only (70 bp).

FIG. 4. Multiplex of the species specific oligonucleotide pairs. Amplified fragments were detected by ethidium bromide staining on a 4% Nusieve agarose gel. Lane 1 shows the 100 bp ladder (Invitrogen). Lane 2 shows the multiplex performance of oligonucleotide pairs specific for the following 14 species: pig, human, cat, Chinese hamster, Rhesus monkey, sheep, horse, Green monkey, rat, dog, mouse, rabbit, goat, and bovine. The template for the reactions consisted of 0.5 ng-1.0 ng mixed DNA contributed from all of the species with primers in the master mix.

FIG. 5. When multiplexed the redesigned primers can detect 12 species in 3 reactions. Amplified fragments were detected by ethidium bromide staining on a 4% Nusieve agarose gel. (A) Master mix I detects 6 species; bovine (lane 2), mouse (lane 3), rat (lane 4), horse (lane 5), Chinese hamster (lane 6), human (lane 7). The master mix was challenged against a mixed template of all species and shows positive bands against them all (lane 1). Lane 8 shows the 100 bp ladder (Invitrogen). Note that the internal control is visible at the bottom of every lane. (B) Master mix II detects 3 species; green monkey (lane 2), rhesus monkey (lane 3) and human (lane 4). The master mix was challenged against a mixed template of all of the species and shows positive bands against them all (lane 1). Lane 5 shows the 100 bp ladder. Note that the internal control is visible at the bottom of every lane with mix II. (C) Master mix III detects 5 species; goat (lane 2), rabbit (lane 3), dog (lane 4), cat (lane 5) and human (lane 6). The master mix was challenged against a mixed template of all species and shows positive bands against them all (lane 1). Lane 7 shows the 100 bp ladder. Note that the internal control is visible at the bottom of every lane.

FIG. 6. Detection of interspecies contamination using small ratios of contaminating cells. Mouse cell line (ATCC# CCL-1) and human cell line (ATCC# CCL-2) were mixed together to produce a variety of fixed ratios (as shown in Table 2). Multiplex mixes I, II, and III containing various combinations of species specific oligonucleotides were then challenged to detect small numbers of “contaminating” cells against a large background. The first three lanes show the amplification of a mixed template where X cell from Y cell lines comprising all 11 species were mixed together and used as a positive control for these specific combinations of oligonucleotides. I; amplification of positive control with master mix I, II; amplification of positive control with master mix II, III; amplification of positive control with master mix III. Panels A-K; amplification of mixed templates as shown in Table 2 (A-K). For all panels PCR results with mix I are shown in the lane 1, mix II in the second lane, and mix III in the third lane. The 100 bp marker (Invitrogen) is shown surrounding each panel. The human and mouse template mixtures vary across panels as follows: (A) Human:mouse 1:99, (B) human:mouse 5:95, (C) human:mouse 10:90, (D) human:mouse 20:80, (E) human:mouse 100:0, (F) human:mouse 80:20, (G) human:mouse 90:10, (H) human:mouse 95:5, (I) human:mouse 99:1, (J) human:mouse 0:100, (K) human:mouse 50:50.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims.

For clarity of disclosure, and not by way of limitation, the detailed description of the invention is divided into the subsections that follow.

This invention provides a simple, rapid, and useful method for differentiating cells from various organisms. This invention enables a rapid, simple and useful method to apply the species- (and genus)-specific probes (fragments and/or oligonucleotides) and/or amplification primers for the identification of cells of particular cell lines. Ultimately, these probes (fragments and/or oligonucleotides) and/or amplification primers can be used in PCR assays so that the identity of the cell line can be confirmed.

A. Definitions:

As used herein, “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “polymerase chain reaction (PCR)” refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and a heat-stable DNA polymerase, e.g. Taq DNA polymerase. In a series, e.g. 30, of temperature cycles, the target DNA is repeatedly denatured (e.g. around 90° C.), annealed to the primers (e.g., at 50-60° C.) and a daughter strand extended from the primers (e.g. 72° C.). As the daughter strands themselves act as templates for subsequent cycles, DNA fragments matching both primers are amplified exponentially, rather than linearly. The original DNA need thus be neither pure nor abundant, and the PCR reaction has accordingly become widely used not only in research, but in clinical diagnostics and forensic science.

As used herein, “Multiplex PCR” refers to variant of conventional polymerase chain reaction that uses at least two or more primer pairs to amplify different stretches of a target DNA molecule simultaneously.

As used herein, “primers” refer to oligonucleotides of about 3 bp to about 100 bp in length used for initiating polymerase chain reaction.
Forward: a primer that may bind to one of the two complementary anti-parallel DNA strands.
Reverse: primer that may bind to a strand that is complementary to the strand to which the forward primer binds.

Sample: A biological sample such as cell culture, saliva, stools, body fluids, urine, blood, tissue biopsy, gastric biopsy, gastrointestinal tissue, tumor cells, mucus secretions, dental plaque, whole cell isolates, and other biological tissues, meat products, food products, microbiological isolates, and environmental samples such as air, soil and water.

As used herein, “nested PCR” refers to a PCR in which specificity is improved by using two sets of primers sequentially. An initial PCR is performed with the “outer” primer pairs, then a small aliquot is used as a template for a second round of PCR with the “inner” primer pair.

As used herein, “reverse transcription PCR or RT-PCR” refers to PCR in which the starting template is RNA, implying the need for an initial reverse transcriptase step to make a DNA template. Some thermostable polymerases have appreciable reverse transcriptase activity; however, it is more common to perform an explicit reverse transcription, inactivate the reverse transcriptase or purify the product, and proceed to a separate conventional PCR.

As used herein, “said target sequences are comparably amplified” means that the degree of the amplification of the different target sequences in the multiplex PCR achieve the desired uniformity. Normally, the difference among the amplification level of different target sequences should be within about 50% from each other. Preferably, the difference among the amplification level of the different target sequences should be within about 40%, 30%, 20%, 10%, 5%, 4%, 3%, 2%, 1% from each other. More preferably, the different target sequences are amplified to the same degree.

As used herein, “nucleic acid(s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotide primers, locked nucleic acid (LNA) and the like.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity.

“Complementary or matched” also means that two nucleic acid sequences can hybridize under low, medium or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity.

Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows: 1) high stringency: O.1×SSPE (or O.1×SSC), 0.1% SDS, 65° C.; 2) medium stringency: 0.2×SSPE (or 1.0×SSC), 0.1% SDS, 50° C. (also referred to as moderate stringency); and 3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “melting temperature” (“Tm”) refers to the midpoint of the temperature range over which nucleic acid duplex, i.e., DNA:DNA, DNA:RNA, RNA:RNA, PNA: loDNA, LNA:RNA and LNA: DNA, etc., is denatured.

As used herein, “sample” refers to anything which includes a target nucleic acid to be amplified by PCR, e.g. multiplex PCR.

As used herein, the phrase “increasing the specificity” of an assay means reducing the frequency or likelihood of false positive assay results. The specificity of an assay is “increased” relative to another assay if there are at least about 10% fewer false positive assay results, and preferably at least about 20%, 30%, 50%, 75%, 90% or more, up to and including 100% fewer (no false positives) in that assay relative to the other.

As used herein, the term “hybridizes,” when used in reference to an oligonucleotide primer, refers to the formation of a hydrogen-bonded base paired duplex with a nucleic acid having a sequence sufficiently complementary to that of the oligonucleotide primer to permit the formation of such a duplex under the conditions used. As the term is used herein, exact complementarity between an oligonucleotide primer and a nucleic acid sequence is not required, with mismatches permitted as long as the resulting duplex is a substrate for extension by a template-dependent nucleic acid extending enzyme. A nucleic acid sequence is “sufficiently complementary” to an oligonucleotide primer if the primer can form a duplex with a molecule comprising the nucleic acid sequence at 55° C. that can be extended by at least one nucleotide by a template-dependent nucleic acid extending enzyme (e.g., a polymerase in a solution comprising 10 mM Tris-HCl, pH 8.8, 50 mM KCl, 2.0 mM MgCl₂, and 200 μM each of dATP, dCTP, dGTP, and dTTP).

As used herein, the phrase “standard conditions,” when used in reference to nucleic acid hybridization, refers to incubation at 55° C. in a buffer containing 15 mM Tris-HCl, pH 8.0, 50 mM KCl, and 2.5 mM MgCl₂, or its equivalent. Oligonucleotide primer molecules hybridized to a template nucleic acid (e.g., a Mycoplasma 16S rRNA gene or an internal amplification control template) under these conditions will be extended by at least one nucleotide by a template-dependent nucleic acid extending enzyme provided that the 3′-terminal two nucleotides of the primer are base paired to the template.

As used herein, the phrase “does not base pair with” or “is mismatched” means that a given sequence of nucleotides on an oligonucleotide primer does not form complementary hydrogen bonds with an adjacent nucleotide sequence on a nucleic acid molecule. As the phrase is used herein, when one or more 3′-terminal nucleotides on an oligonucleotide primer “do not base pair” with a template nucleic acid molecule, a template-dependent nucleic acid extending enzyme will not extend the primer by one nucleotide or more under annealing and polymerization conditions as follows: 10 μCi of each of ³³P-labeled dATP, dCTP, dGTP, and dTTP (>1000 Ci/mMole), 1× Taq polymerase buffer (10 mM Tris-HCl, pH 8.8, 50 mM KCl, 1.5 mM MgCl₂, 0.001% (w/v) gelatin; or its equivalent), 100 nM of primer, 2.0 mM MgCl₂, 100 fmol template and 0.04 U/μl of Taq200™ polymerase (Stratagene #600197-51); the mixture is heated at 94° C. for 30 seconds, annealing is performed at 55° C. for 30 seconds, and polymerization is performed at 72° C. for one minute. The presence of one or more labeled species detected by autoradiography when the reaction products are separated on polyacrylamide gel demonstrates the extension of the primer. If there are no labeled species, then the terminal nucleotide(s) of the primer “does not base pair with” the template. Alternatively, when the sequence of a potential contaminating template, e.g., an E. coli 16S rRNA gene sequence, is known, one can manually or via computer (e.g., using BLAST, with default parameters) align a given primer sequence with the contaminating template sequence. If one or more (e.g., one, two, three) of the 3′-terminal three nucleotides of the primer are not complementary to the template, they “do not base pair” with the template.

As used herein, the phrase “amplification control template” refers to a double- or single-stranded nucleic acid molecule that is added to a nucleic acid amplification reaction to serve as a control for the activity of the template-dependent nucleic acid extending enzyme used in such reaction. Various suitable control templates are known in the art. Amplification of the amplification control template can be distinguished from amplification of the target template by melting temperature or product length.

As used herein, the term “set” refers to a group of at least two. Thus, a “set” of oligonucleotide primers comprises at least two oligonucleotide primers.

As used herein, an “oligonucleotide” and a “primer” are used interchangeably in their most general sense to include any length of nucleotides which, when used for amplification purposes, can provide a free 3′ hydroxyl group for the initiation of DNA synthesis by a DNA polymerase, either using a RNA or a DNA template. DNA synthesis results in the extension of the primer to produce a primer extension product complementary to the nucleic acid strand to which the primer has hybridized. Generally, the primer comprises from 3 to 100 nucleotides, preferably from 5 to 50 nucleotides and even more preferably from 10 to 35 nucleotides. Primers are often selected to be any number of nucleotides between 10 and 25 nucleotides or more in length. The primers of the present invention may be synthetically produced by, for example, the stepwise addition of nucleotides, or may be fragments, parts, portions or extension products of other nucleic acid molecules. The term primer is used generally to encompass both strands of a given sequence (i.e., a given sequence and its complementary sequence).

As used herein, “complementary” refers to the broad concept of sequence complementarity between regions of two polynucleotide strands or between two nucleotides through base-pairing. It is known that an adenine nucleotide is capable of forming specific hydrogen bonds (“base pairing”) with thymine or uracil. Similarly, it is known that a cytosine nucleotide is capable of base pairing with a guanine nucleotide. This hydrogen bonding is the basis of the hybridization mentioned in this document.

As used herein, the phrase “extension product” refers to the nucleic acid product of an extension reaction catalyzed by a template-dependent nucleic acid extending enzyme. An “extension product” has been extended by at least one nucleotide by a template-dependent nucleic acid extending enzyme.

As used herein, the phrase “detectably different in size or sequence” means that the extension or amplification product formed by enzymatic extension or amplification of a nucleic acid template can be distinguished from the extension or amplification product of a target nucleic acid on the basis of a difference in size or sequence using techniques known to those of skill in the art or described herein. Conditions are well known for the separation of nucleic acids differing by as little as one nucleotide in length. Thus, the phrase “detectably different in size or sequence” means that a molecule differs by at least one nucleotide in length from another. It is preferred, however, that molecules of “detectably different” size differ by more than one nucleotide, e.g., by at least 10 nucleotides, 50 nucleotides, 100 nucleotides, or more. Alternatively, molecules of different sequence can be distinguished, e.g., on the basis of an enzymatic cleavage site or a binding site for a ligand that is present on one nucleic acid molecule but not on the other. Likewise, they can be distinguished based on their respective melting temperatures. This technique can be advantageous in the context of QPCR reactions because differences in melting temperatures can be determined without the need to prepare and run separate reactions, gels, etc. Thus, in the context of QPCR reactions, this technique can be easier to perform, generate less waste, and provide results faster than other techniques for detecting different sizes or sequences. Such molecules are thus of “detectably different” sequence.

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably, the substantially similar nucleotide sequence encodes the polypeptide encoded by the reference nucleotide sequence. The percentage of identity between the substantially similar nucleotide sequence and the reference nucleotide sequence (number of complementary bases in the complementary sequence divided by total number of bases in the complementary sequence) desirably is at least 80%, more desirably 85%, preferably at least 90%, more preferably at least 95%, most preferably at least 99%.

The terms “identical” or “percent identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

“Substantially identical,” in the context of two nucleic acid or protein sequences, refers to two or more sequences or subsequences that have at least 60%, preferably 80%, more preferably 90-95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y., which is hereby incorporated by reference. Generally, highly stringent hybridization and wash conditions are selected to be about 5 degrees C. lower than the thermal melting point (Tm) for the specific sequence at a defined ionic strength and pH. Typically, under “stringent conditions” a probe will hybridize to its target subsequence, but to no other sequences.

The T.sub.m is the temperature (under defined ionic strength and pH) at which 50% of the target sequence hybridizes to a perfectly matched probe. Stringent conditions are selected to be equal to the Tm for a particular probe. An example of stringent hybridization conditions for hybridization of complementary nucleic acids which have more than 100 complementary residues on a filter in a Southern or northern blot is 50% formamide with 1 mg of heparin at 42 degrees C., with the hybridization being carried out overnight. An example of highly stringent wash conditions is 0.15 M NaCl at 72 degrees C. for about 15 minutes. An example of stringent wash conditions is a 0.2 times SSC wash at 65 degrees C. for 15 minutes (see, Sambrook, infra, for a description of SSC buffer). Often, a high stringency wash is preceded by a low stringency wash to remove background probe signal. An example of medium stringency wash for a duplex of, e.g., more than 100 nucleotides, is 1 times SSC at 45 degrees C. for 15 minutes. An example of low stringency wash for a duplex of, e.g., more than 100 nucleotides, is 4-6 times SSC at 40 degrees C. for 15 minutes. For short probes (e.g., about 10 to 50 nucleotides), stringent conditions typically involve salt concentrations of less than about 11.0M Na ion, typically about 0.01 to 1.0 M Na ion concentration (or other salts) at pH 7.0 to 8.3, and the temperature is typically at least about 30 degrees C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPOsub4, 1 mM EDTA at 50 degrees C. with washing in 2 times SSC, 0.1% SDS at 50 degrees C., more desirably in 7% sodium dodecyl sulfate (SDS), 0.5 M NaPOsub4, 1 mM EDTA at 50 degrees C. with washing in 0.1 times SSC, 0.1% SDS at 50 degrees C., more desirably still in 7% sodium dodecyl sulfate (SDS), 0.5 M NaPOsub4, 1 mM EDTA at 50 degrees C. with washing in 0.5 times SSC, 0.1% SDS at 50 degrees C., preferably in 7% sodium dodecyl sulfate (SDS), 0.5 M NaPOsub4, 1 mM EDTA at 50 degrees C. with washing in 0.1 times SSC, 0.1% SDS at 50 degrees C., more preferably in 7% sodium dodecyl sulfate (SDS), 0.5 M NaPOsub4, 1 mM EDTA at 50 degrees C., with washing in 0.1 times SSC, 0.1% SDS at 65 degrees C.

As used herein, “assessing PCR products” refers to quantitative and/or qualitative determination of the PCR products, and also of obtaining an index, ratio, percentage, visual or other value indicative of the level of the PCR products. Assessment may be direct or indirect and the chemical species actually detected need not, of course, be the PCR products themselves, but may, for example, be a derivative thereof, or some further substance.

The PCR products can be assessed by any suitable methods. For example, the PCR products can be assessed via agarose gel electrophoresis. When assessed by the agarose gel electrophoresis, the difference of the length of the PCR products is preferably more than about 30 base pairs (bp). More preferably, the difference of the length of the PCR products is from about 30 bp to about 50 bp. The PCR products can be assessed by other methods such as polyacrylamide gel electrophoresis and capillary electrophoresis.

B. Development of Species-Specific, Genus-Specific DNA Probes and Amplification Primers for Organisms

The present invention relates to PCR based assay methods and materials for detecting and identifying contaminating cells from mammalian cell lines, as well as their related species and genera, that may be present as contaminates in a cell culture and can be detected in a test sample taken from such a cell culture.

In one embodiment, the invention is directed to a method using probes (DNA fragments and/or oligonucleotides) and/or amplification primers which are specific and sensitive for determining the presence and/or amount of nucleic acids from contamination cells of cell lines in any sample suspected of containing said nucleic acid from contaminating cells of cell lines, wherein said contaminating cells of cell lines nucleic acid or variant or part thereof comprise a selected target region hybridizable with said probes or primers; said method comprising the steps of contacting said sample with said probes or primers and detecting the presence and/or amount of hybridized probes and/or amplified products as an indication of the presence and/or amount of contaminating cells. In a preferred embodiment, the contaminating cells are from a mammalian cell line. In a more preferred embodiment, mammalian cell lines are selected from the group consisting of human, mouse, rat, cat, dog, bovine, pig, sheep, goat, horse, Chinese hamster, Green monkey, Rhesus monkey or rabbit.

In one embodiment, the method of the invention is performed directly on a sample obtained from a cell culture, environment or food. In another embodiment, the method is performed directly on a sample consisting of one or more cell lines. In another embodiment, cell line nucleic acid is amplified by a method selected from the group consisting of: a) polymerase chain reaction (PCR), b) ligase chain reaction, c) nucleic acid sequence-based amplification, d) self-sustained sequence replication, e) strand displacement amplification, f) branched DNA signal amplification, g) nested PCR, h) microarray, and I) multiplex PCR. In another embodiment, nucleic acid from cells of a contaminating cell line is amplified by PCR. In another embodiment the PCR protocol is modified to determine, within one hour, the presence of said the target nucleic acid present in a culture suspected of containing contaminating cells by performing for each amplification cycle an annealing step of only one second at 55° C. and a denaturation step of only one second at 95° C. without any elongation step.

In another embodiment, the invention is directed to a method for the detection, identification and/or quantification the presence of contaminating mammalian cells of cell lines directly from a test sample or from colonies taken from a culture or cell line suspected of being contaminated or subject to contamination, which comprises the following steps: a) depositing and fixing on an inert support or leaving in solution the contaminating mammalian cell line DNA of the sample, and lysing in situ said sample to release the DNA from the contaminating cells of mammalian cell lines, said contaminating mammalian cell lines DNA being in a substantially single stranded form; b) contacting said single stranded DNA with a probe, said probe comprising at least one single stranded nucleic acid which nucleotidic sequence is selected from the group consisting of SEQ ID NO:3, SEQ ID NO: 4, SEQ ID NO: 5, SEQ ID NO: 6, SEQ ID NO: 7 (or any other sequence from Table 1), a sequence complementary thereof, a part thereof and a variant thereof, which specifically anneals with contaminating mammalian cell line DNA or representatives of the DNA of contaminating mammalian cell lines, under conditions such that the nucleic acid of said probe can selectively hybridize with the DNA of the contaminating cells of mammalian cell lines, whereby a hybridization complex is formed, said complex being detected by labelling means, the label being present on said probe or the label being present on a first reactive member of said labelling means, said first reactive member reacting with a second reactive member present on said probe; and c) detecting the presence or the intensity of said label on said inert support or in said solution as an indication of the presence and/or amount of a specific contaminating mammalian cell lines in said test sample. In a preferred embodiment, the probe for detecting nucleic acid sequences from contaminating mammallian cell lines is selected from the group consisting of SEQ ID numbers that are listed in Table 1 and a sequence complementary thereof.

In another embodiment, the invention is directed to a method for detecting the presence and/or amount of contaminating cells, which may be present in a mammalian cell line or culture, by testing a sample, therefrom, wherein the test which comprises the following steps: a) treating said sample with an aqueous solution containing at least one pair of oligonucleotide primers having at least 12 nucleotides in length, one of said primers being capable of hybridizing selectively with one of the two complementary strands of the DNA isolated from or present in the contaminating cells of mammalian cell lines that contains a target sequence, and the other of said primers being capable of hybridizing with the other of said strands so as to form an extension product which contains the target sequence as a template, said at least one pair of primers being chosen from within one of the sequences that are listed in Table 1; b) synthesizing an extension product of each of said primers which extension products contain the target sequence, and amplifying said target sequence, if any, to a detectable level; and c) detecting the presence and/or amount of said amplified target sequence as an indication of the presence and/or amount of contaminating cells in mammalian cell lines in said test sample. In a preferred embodiment, the method uses at least one pair of primers selected from the sequences that are listed in Table 1. In a preferred embodiment, the method uses at least one pair of primers that is selected from the group consisting of: the sequences that are listed in Table 1.

In another embodiment, the invention is directed to a nucleic acid having the nucleotide sequence of any one of SEQ ID Nos that are listed in Table 1, a part thereof and variants thereof which, when in single stranded form, specifically hybridize with a target contaminating mammalian cell or cell line DNA as a probe or as a primer.

According to this invention, the oligonucleotides, primers or probes may be derived from either strand of the duplex DNA. The oligonucleotides, primers or probes may consist of the bases A, G, C, or T or analogs and they may be degenerated at one or more chosen nucleotide position(s). In one embodiment, the oligonucleotides, primers or probes may be of any suitable length and may be selected anywhere within the DNA sequences from proprietary fragments.

In another embodiment, the invention is directed to an oligonucleotide having a nucleotidic sequence of any one of SEQ ID Nos that are listed in Table 1.

In another embodiment, the invention is directed to a recombinant plasmid comprising a nucleic acid of any one of SEQ ID Nos that are listed in Table 1.

In another embodiment, the invention is directed to a recombinant host which has been transformed by a recombinant plasmid comprising a nucleic acid of any one of the SEQ ID Nos that are listed in Table 1. In a preferred embodiment, the recombinant host is Escherichia coli.

The various species-specific and genus-specific PCR assays which are embodiments of the present invention are all specific. For the PCR assays specific to species or genus, this means that DNA isolated from cells of a wide variety of species, other than that from the target species or genus and selected from Table 1, could not be amplified. For the PCR assay specific to cells from a human cell line, it means there was no amplification with genomic or subgenomic DNA from cells of other species (e.g., mouse, rat, cat, dog, bovine, pig, sheep, goat, horse, Chinese hamster, Green monkey, Rhesus monkey and rabbit).

The present invention relates to assay methods and materials for detecting and identifying different cells or nucleic acid molecules from species such as human, mouse, rat, cat, dog, bovine, pig, sheep, goat, horse, Chinese hamster, Green monkey, Rhesus monkey and rabbit that contaminate a test sample, such as a sample from a cell culture.

The present invention provides a method for detecting or identifying different cells or nucleic acid molecules from species such as human, mouse, rat, cat, dog, bovine, pig, sheep, goat, horse, Chinese hamster, Green monkey, Rhesus monkey and rabbit, present as a contaminant in a cell culture, comprising the following steps: a) if necessary, extracting nucleic acids from a sample such as cell lines, biological products or human tissue or serum; b) amplifying target DNA of different species such as human, mouse, rat, cat, dog, bovine, pig, sheep, goat, horse, Chinese hamster, Green monkey, Rhesus monkey and rabbit using more than one proper primer and c) detecting signals generated from the hybridization reaction.

From the detected signals in the step c), the existence of different cells or nucleic acid from species such as human, mouse, rat, cat, dog, bovine, pig, sheep, goat, horse, Chinese hamster, Green monkey, Rhesus monkey and rabbit and its related strains in the sample can be determined.

The present inventors carried out a sequence analysis of the cytochrome c oxidase I (COI) and/or cytochrome b to obtain species-specific and genus-specific oligonucleotides for detecting the presence of cells from various mammals and mammals-related species which can be a basis of developing a specific and sensitive hybridization assay. In one embodiment, the present invention provides nucleic acids that can be used in detecting the presence of cells from cell lines of various mammal species and genera. The nucleic acids can be oligonucleotides that can function as primers for PCR reactions. The nucleic acids can also be primers or genomic or sub-genomic nucleic acids that can be used as controls for monitoring the progression, specificity, and/or sensitivity of the methods of the invention.

In one embodiment, oligonucleotides according to the invention can specifically hybridize to nucleic acids of human, mouse, rat, cat, dog, bovine, pig, sheep, goat, horse, Chinese hamster, Green monkey, Rhesus monkey and rabbit. Suitable primers can be designed by those of skill in the art based on known nucleic acid sequences of this invention to identify the cells or nucleic acid of human, mouse, rat, cat, dog, bovine, pig, sheep, goat, horse, Chinese hamster, Green monkey, Rhesus monkey and rabbit in a cell culture.

It is another embodiment of the present invention to provide a microarray comprising genus-specific and species-specific oligonucleotides for detecting cells of human, mouse, rat, cat, dog, bovine, pig, sheep, goat, horse, Chinese hamster, Green monkey, Rhesus monkey and rabbit in a cell culture and its related species and genera as oligonucleotides.

It is another embodiment of the present invention to provide a method for detecting cells of human, mouse, rat, cat, dog, bovine, pig, sheep, goat, horse, Chinese hamster, Green monkey, Rhesus monkey and rabbit in a cell culture and its related species and strains using a PCR based assay such as multiplexed PCR and microarray.

It is another embodiment of the present invention to provide methods (i.e., assays) that detect more than one species of a given genus. That is, the present invention contemplates detecting the cells or nucleic acid of multiple members of the cells of human, mouse, rat, cat, dog, bovine, pig, sheep, goat, horse, Chinese hamster, Green monkey, Rhesus monkey and rabbit in the same assay. Therefore, the present invention targets a genomic sequence that is well conserved among the human, mouse, rat, cat, dog, bovine, pig, sheep, goat, horse, Chinese hamster, Green monkey, Rhesus monkey and rabbit.

For the purposes of the present invention, oligonucleotides of human, mouse, rat, cat, dog, bovine, pig, sheep, goat, horse, Chinese hamster, Green monkey, Rhesus monkey and/or rabbit, were identified wherein attention was primarily focused on cytochrome c oxidase I (COI) and/or cytochrome b subsequences. Oligonucleotides synthesis can be carried out by any known method. For example, oligonucleotides can be produced using cyanoethyl phosphoramidite chemistry, ammonium hydroxide deprotection, and desalting by gel filtration chromatography. If desired, primers can be further purified by high performance liquid chromatography (HPLC), polyacrylamide gel electrophoresis (PAGE), or any other method known to those of skill in the art.

Nucleic acids other than oligonucleotides are also part of this invention. These nucleic acids can be used as controls for monitoring various aspects of the methods of the present invention. The control nucleic acids can be cytochrome c oxidase I (COI) and/or cytochrome b nucleic acids, which can be used as positive controls to confirm that the methods and oligonucleotides are suitable for amplification and detection of cytochrome c oxidase I (COI) and/or cytochrome b subsequences nucleic acids. The control nucleic acids can also be other mammalian nucleic acids or eukaryotic nucleic acids. These other nucleic acids can be used to confirm the specificity of the primers used to amplify and detect cytochrome c oxidase I (COI) and/or cytochrome b nucleic acids and to confirm that no inhibitors of amplification were present in the amplification mixtures. Control nucleic acids can be genomic or sub-genomic nucleic acids.

In the detection method according to the present invention, the sample may be a biological drug, cell line, or human tissues, mammalian tissues, or serum.

It is another embodiment of the present invention to provide a kit for identifying cells from cell lines of human, mouse, rat, cat, dog, bovine, pig, sheep, goat, horse, Chinese hamster, Green monkey, Rhesus monkey and rabbit as well as its related species and genera individually or simultaneously, comprising genus-specific and species-specific oligonucleotides for cells of human, mouse, rat, cat, dog, bovine, pig, sheep, goat, horse, Chinese hamster, Green monkey, Rhesus monkey and rabbit and its related species and genera.

The cytochrome c oxidase I (COI) and/or cytochrome b subsequence of the target DNA of the present invention can be used indirectly for designing probes or primers used for identifying cells and/or nucleic acid from human, mouse, rat, cat, dog, bovine, pig, sheep, goat, horse, Chinese hamster, Green monkey, Rhesus monkey and rabbit species as well as related species and genera, or directly for identifying cells and/or nucleic acid from human, mouse, rat, cat, dog, bovine, pig, sheep, goat, horse, Chinese hamster, Green monkey, Rhesus monkey and rabbit species as well as related species and genera via PCR amplification.

According to another aspect of the present invention, there is provided an oligonucleotide for genus-specific, species-specific or human, mouse, rat, cat, dog, bovine, pig, sheep, goat, horse, Chinese hamster, Green monkey, Rhesus monkey and rabbit, comprising a sequence selected from SEQ ID Nos. as listed in Table (1) or its complementary sequence.

According to one embodiment of the invention, the oligonucleotides can be used as primers for PCR amplification in order to identify cells or nucleic acid from human, mouse, rat, cat, dog, bovine, pig, sheep, goat, horse, Chinese hamster, Green monkey, Rhesus monkey and rabbit cell lines as well as their related species or genera or as probes for hybridization reaction in order to identify human, mouse, rat, cat, dog, bovine, pig, sheep, goat, horse, Chinese hamster, Green monkey, Rhesus monkey and rabbit cell lines as well as their related species and genera.

The purifying step of the nucleic acid can be performed using a conventional DNA or RNA purification method or kit. According to another aspect of the present invention, there is provided a kit for identification of contaminated cell lines including, but not limited to, contaminated mammalian cell lines, comprising more than one oligonucleotide selected from species-specific and/or genus-specific oligonucleotides for identifying contaminating cells from, but not limited to, mammalian cell lines such as human, mouse, rat, cat, dog, bovine, pig, sheep, goat, horse, Chinese hamster, Green monkey, Rhesus monkey and rabbit according to the above present invention.

The present invention provides kits for detecting the contamination of cell lines by cells from various mammalian genera and species which may be present in a sample. In its most basic form, the kit of the invention can comprise one or more nucleic acids or compositions containing such nucleic acids provided by the present invention. The kits can comprise the components in a single package or in more than one package within the same kit. Where more than one package is included within a kit, each package can independently contain a single component or multiple components, in any suitable combination. As used herein, a combination of two or more packages or containers in a single kit is referred to as “in packaged combination”. The kits and containers within the kits can be fabricated with any known material. For example, the kits themselves can be made of a plastic material or cardboard. The containers that hold the components can be, for example, a plastic material or glass. Different containers within one kit can be made of different materials. In embodiments, the kit can contain another kit within it. For example, the kit of the invention can comprise a kit for purifying nucleic acids.

In general, the kits can comprise, in a single package or in packaged combination, two or more oligonucleotides (primers), reagents and/or other components for performing the methods of the invention, nucleic acid templates for use as positive controls or specificity controls, or combinations of two or more of these.

In similar embodiments, the kit according to the invention comprises at least two primers, where each of the primers has a sequence comprising a SEQ ID NO (the sequence can be selected from sequences that are encompassed by this invention), where each primer present in the kit has a sequence that differs from each other primer sequence in the kit. In these embodiments, the kit comprises two primers, one comprising the sequence of SEQ ID NO:1 and the other comprising the sequence of SEQ ID NO:2 (for example—the sequence can be selected from sequences of this invention). In other embodiments, the kit comprises three primers, one comprising the sequence of SEQ ID NO:1, the second comprising the sequence of SEQ ID NO:2, and the third primer comprising the sequence of SEQ ID NO:3, SEQ ID NO:4, or SEQ ID NO:5 (for example). In an exemplary embodiment, the kit comprises a primer comprising the sequence of SEQ ID NO:1, a primer comprising the sequence of SEQ ID NO:2, a primer comprising the sequence of SEQ ID NO:3, and a primer comprising the sequence of SEQ ID NO:4. In yet another exemplary embodiment, the kit comprises a primer comprising the sequence of SEQ ID NO:1, a primer comprising the sequence of SEQ ID NO:2, a primer comprising the sequence of SEQ ID NO:3, a primer comprising the sequence of SEQ ID NO:4, and a primer comprising the sequence of SEQ ID NO:5. Other primer sequences of this invention may be used as part of this invention. In certain embodiments, all of the primers provided in the kit are provided in a single container, whereas in other embodiments, they are provided in at least two separate containers, alone or in combination with one or more other primer. In a specific embodiment, the kit can comprise a set of two or more, and preferably four, five or any of those primers that are encompassed by this invention as described herein, that ecognize and amplify specific cytochrome c oxidase I (COI) and/or cytochrome b subsequences. In the present invention, the probes may be a combination of more than one probe capable of simultaneously detecting many contaminations of the tested cell lines, including but not limited to, contaminating mammalian cell lines and their related mammalian genera and mammalian species from a single sample. Practically, the probes are optimized to simultaneously hybridize with multiple target DNAs of cell lines of mammalian and as well as cell lines of mammalian related genera and species under the same hybridization and washing conditions.

Accordingly, the invention provides a kit comprising at least one container holding a composition comprising at least one oligonucleotide primer, each of these primers having a sequence comprising the sequence of one or more of the sequences according to the invention. For example, the kit can comprise a container containing a primer having a sequence comprising SEQ ID NO:1, and a container containing a primer having a sequence comprising SEQ ID NO:2. Likewise, other containers can be provided that contain a primer having a sequence comprising SEQ ID NO:3, a primer having a sequence comprising SEQ ID NO:4, and a primer having a sequence comprising SEQ ID NO:5 and so on. Alternatively, two or more primers can be contained in one container, the invention not being limited by any particular combination of primers in each container. In one aspect of the invention, containers can be provided that contain primers having all of the sequences that are listed in the invention.

The kit of the invention can comprise primers for the amplification control (AC). The AC primers may be contained in a container separate from the other components of the kit. In another embodiment, the AC primers are contained in the same container as the mammalian primers. In another possible embodiment, the AC primers are contained in the same container as the AC template nucleic acid. Similarly, the AC primers, the AC template nucleic acid, and at least one mammalian primer may be contained in the same container. In one embodiment, the mammalian primer is selected from the group of human, mouse, rat, cat, dog, bovine, pig, sheep, goat, horse, Chinese hamster, Green monkey, Rhesus monkey and rabbit primers according to the above present invention. In exemplary embodiments, the AC primers individually comprise the sequences of one or more the sequences of this invention.

The kit of the invention can comprise purified mammalian nucleic acids, including but not limited to, purified nucleic acid that is isolated from human, mouse, rat, cat, dog, bovine, pig, sheep, goat, horse, Chinese hamster, Green monkey, Rhesus monkey and rabbit according to the above present invention.

In another embodiment of the present invention, the concentration of primer mix may be balanced. Each primer solution may be prepared at a concentration of 10 pmol/μl. Equal volume of the two primers or more may be added to the PCR mix. The amplification of individual contaminant may be tested and according to the results the concentration of primers may be adjusted for subsequent experiments. Primers corresponding to multiple contaminants may be mixed and the PCR may be performed. Depending on the results, the final primer concentration may be adjusted. For each primer, the concentration may be about 0.05-0.1 pmol/μl for the PCR reaction. In another embodiment of the invention, the concentration of each primer may be about 0.1-1.0 pmol/μl. The final concentrations of primers may be tested under different PCR conditions to make sure that the primer mix would work well under a range of conditions, such as the amount of DNA template, polymerase, Mg++ concentration, and a range of annealing temperatures and the like.

These nucleic acids of the present invention can be genomic or sub-genomic nucleic acids. In exemplary embodiments, the kits comprise a container containing purified gDNA of mammals, including but not limited to, purified genomic (gDNA) that is isolated from human, mouse, rat, cat, dog, bovine, pig, sheep, goat, horse, Chinese hamster, Green monkey, Rhesus monkey and rabbit.

In other embodiments, a known amount of mammal or mammal related genera and species gDNA is contained in a single container within the kit. In certain cases, two containers, each containing one or the other genomic DNA (gDNA) of mammals, related species and genera, are included in the kit. In certain other cases, a human gDNA and a mouse gDNA are included in a single container within the kit. In other embodiments, gDNA from each mammalian related species and genera is provided in the kit, either in separate containers or together in a single container.

The kit of the invention can comprise one or more components useful for amplifying target sequences of mammals, including but not limited to, target sequences of human, mouse, rat, cat, dog, bovine, pig, sheep, goat, horse, Chinese hamster, Green monkey, Rhesus monkey and rabbit according to the above present invention.

In other embodiments, some or all of the reagents and supplies necessary for performing PCR are included in the kit, non-limiting examples of reagents are buffers (e.g., a buffer containing Tris®, HEPES® and the like), salts, and a template-dependent nucleic acid extending enzyme (such as a thermostable enzyme, such as Taq polymerase), a buffer suitable for activity of the enzyme, and additional reagents needed by the enzyme, such as dNTPs, dUTP, and/or a UDG enzyme. In other embodiments, the kit comprises Brilliant® SYBR® Green QPCR Master Mix (Catalog # 600548, Stratagene, La Jolla, Calif.). A non-limiting example of supplies is reaction vessels (e.g., microcentrifuge tubes).

The kit can comprise at least one dye for detecting nucleic acids, including, but not limited to, dsDNA. The kit may comprise a sequence-non-specific dye that detects dsDNA, such as SYBR® Green dye (Molecular Probes, Eugene, Oreg.). The dye is preferably contained alone in a separate container. In other embodiments, the dye is provided as a concentrated stock solution, for example, as a 50× solution. In other embodiments, the kit comprises a passive reference dye. The passive reference dye can be included in the kit alone in a separate container. The passive reference dye can be provided as a concentrated stock solution, for example, as a 1 mM stock solution. A non-exclusive exemplary passive reference dye is ROX dye. In other embodiments, the kit contains either a DNA-detecting dye or a passive reference dye. In other embodiments, the kit contains both a DNA-detecting dye and a passive reference dye.

The kit can also comprise one or more components useful for purifying nucleic acids. These components are particularly suited for purifying nucleic acid isolated from mammals, including but not limited to, nucleic acid that is isolated from human, mouse, rat, cat, dog, bovine, pig, sheep, goat, horse, Chinese hamster, Green monkey, Rhesus monkey and rabbit cell lines according to the above present invention.

The components can be, among other things, reagents and supplies that can be used to purify nucleic acids. Non-limiting examples of such reagents and supplies include a DNA binding solution, a wash buffer, and containers, such as microcentrifuge tubes, for collection of binding solutions, wash buffers, and purified nucleic acids. The components can also contain a resin, gel, or other substance that is useful for purifying nucleic acids. In one embodiment, the kit comprises the components of the StrataPrep® PCR Purification Kit (Catalog # 400771, Stratagene, La Jolla, Calif.).

In one embodiment, this invention provides a microarray comprising more than one primer selected from genus-specific or species-specific primers for identifying cells of human, mouse, rat, cat, dog, bovine, pig, sheep, goat, horse, Chinese hamster, Green monkey, Rhesus monkey and rabbit wherein the primer(s) is attached to a support.

The microarray according to the present invention can be used for simultaneously identifying various cell lines of human, mouse, rat, cat, dog, bovine, pig, sheep, goat, horse, Chinese hamster, Green monkey, Rhesus monkey and rabbit, as well as their related species and genera which are known as a contaminant of biological drug or cell lines. The microarray may, also, comprise human, mouse, rat, cat, dog, bovine, pig, sheep, goat, horse, Chinese hamster, Green monkey, Rhesus monkey and rabbit-specific oligonucleotides for identifying human, mouse, rat, cat, dog, bovine, pig, sheep, goat, horse, Chinese hamster, Green monkey, Rhesus monkey and rabbit cell line and its related genera and species as a set attached on a support.

The present invention provides a microarray comprising a set of probes which can simultaneously detect a cell of human, mouse, rat, cat, dog, bovine, pig, sheep, goat, horse, Chinese hamster, Green monkey, Rhesus monkey and rabbit and its related species and genera from a single sample with a single experiment.

According to another embodiment of the present invention, there is provided a method for detecting a cell line of human, mouse, rat, cat, dog, bovine, pig, sheep, goat, horse, Chinese hamster, Green monkey, Rhesus monkey and rabbit, comprising the following steps: a) extracting nucleic acids from a sample; b) amplifying target DNA among the extracted nucleic acids; c) hybridizing the amplified target DNA with probes of the microarray or multiplexed PCR according to the present invention; and d) detecting signals generated from the hybridization reaction.

In one embodiment, this invention provides a microarray comprising more than one oligonucleotide selected from cytochrome c oxidase I (COI) or cytochrome b oligonucleotides for detecting and identifying contaminating cells from various cell lines of mammalian related species and genera.

In one embodiment of this invention, the probes are selected from a group consisting of DNA, RNA, PNA, LNA and HNA.

In one embodiment of this invention, the support is selected from a group consisting of slide glass, plastic, membrane, semiconductive chip, silicon and gel.

In one embodiment, this invention provides a method for detecting the presence of contaminating cells from a mammalian cell line which may improperly be present in another cell line, including another mammalian cell line, comprising the following steps: a) extracting nucleic acids from a sample; b) amplifying target DNA among the extracted nucleic acids; c) hybridizing the amplified target DNA with probes of the microarray according to one or more of the embodiments of this invention; and d) detecting signals generated from the hybridization reaction.

In one embodiment, this invention provides a composition comprising at least one oligonucleotide primer, each of said primer having a sequence comprising any one sequence selected from SEQ ID Nos. ______ as shown in TABLE 1.

In one embodiment, this invention provides a composition, further, comprising a primer having a sequence comprising a sequence selected from the group consisting of sequences that are listed in TABLE 1. In a preferred embodiment, the composition comprises an amplification control nucleic acid and at least one oligonucleotide primer specific for said amplification control nucleic acid. In a preferred embodiment, the composition is further comprising Taq polymerase. In a preferred embodiment, the composition is comprising a dye that can specifically detect double stranded DNA. In a preferred embodiment, the kit, further, comprises genomic or sub-genomic mammalian cell line nucleic acids in a second container. In a preferred embodiment, the kit further comprises an amplification control nucleic acid, wherein said amplification control nucleic acid is present in said first container or in a second container.

In a preferred embodiment, the kit further comprises reagents and supplies for purification of nucleic acids. In a preferred embodiment, the kit further comprises, in packaged combination, SYBR Green dye, an amplification control, a mammalian control, a reference dye, a template dependent nucleic acid extending enzyme, and all reagents and supplies necessary to purify nucleic acids.

In one embodiment, this invention further comprises providing an amplification control nucleic acid and at least two primers that specifically hybridize to the amplification control; amplifying the amplification control; and detecting the product of the amplification control amplifying reaction. In one embodiment, amplifying and detecting of the nucleic acid from a contaminating mammalian cell or cell line and amplifying and detecting of amplification control are performed in a different reaction tube. In one embodiment, this invention comprises amplifying and detecting of purified nucleic acid isolated from contaminating mammalian cells or a cell line and amplifying and detecting of amplification control in the same reaction tube.

In one embodiment, this invention is directed to a hybridization platform comprising more than one oligonucleotide selected from cytochrome c oxidase I (COI) or cytochrome b oligonucleotides for detecting and identifying cells from a contaminating mammalian cell line using probes that are attached on a support. In a preferred embodiment, the probes are any one selected from a group consisting of DNA, RNA, PNA, LNA and HNA. In a preferred embodiment, the support is any one selected from a group consisting of slide glass, plastic, membrane, semiconductive chip, silicon and gel.

In one embodiment, this invention is directed to a method for detecting contaminating cells from a mammalian cell line, comprising the following steps: a) extracting nucleic acids from a sample; b) amplifying target DNA among the extracted nucleic acids; c) hybridizing the amplified target DNA with probes of the hybridization platform according to embodiments of this invention; and d) detecting signals generated from the hybridization reaction. In preferred embodiment, the hybridization platform is selected from the group consisting of PCR, FLAP endonuclease assay, TAQMAN quantitative PCR, SCORPION PCR, multiplex PCR, and microarrays.

In another embodiment, this invention comprises a method of detecting the presence of contaminating mammalian cells from a cell line in a sample, the method comprising: (a) isolating nucleic acid from the sample, (b) performing a multiplex polymerase chain reaction, wherein a plurality of nucleic acid fragments representing a plurality of mammalian cell lines are amplified by a plurality of forward and reverse primers; and (c) determining that a sample is positive for the presence of the contaminating mammalian cells or cell line if a number of identified and amplified nucleic acid fragments are sufficient to suggest the presence of the contaminating mammalian cells or cell line in the sample.

In another embodiment, the contaminating cells of a cell line are selected from the group consisting of human, mouse, rat, cat, dog, bovine, pig, sheep, goat, horse, Chinese hamster, Green monkey, Rhesus monkey and rabbit

In another embodiment, the plurality of primers is selected from the group consisting of primers whose DNA sequences listed in Table 1. In another embodiment, the primers comprise the following characteristics:

(a) about twenty to thirty bases long;
(b) melting temperature of about 60° C.;
(c) GC content of about fifty percent;
(d) minimal dimer formation; and
(e) low frequency of mutations in the primer binding site.

In another embodiment of the invention, the primers are isolated from specific cytochrome c oxidase I

### (COI) and/or cytochrome b subsequences.

In another embodiment, this invention is directed to a method for detecting cross contamination in a sample, the method comprising:

(a) performing a multiplex polymerase chain reaction, wherein a plurality of primers amplify a plurality of mammalian nucleic acid fragments which are selected from the group consisting of nucleic acid molecules of human, mouse, rat, cat, dog, bovine, pig, sheep, goat, horse, Chinese hamster, Green monkey, Rhesus monkey and rabbit.
(b) determining that a sample is positive for human, mouse, rat, cat, dog, bovine, pig, sheep, goat, horse, Chinese hamster, Green monkey, Rhesus monkey or rabbit if a number of amplified fragments is sufficient to detect the human, mouse, rat, cat, dog, bovine, pig, sheep, goat, horse, Chinese hamster, Green monkey, Rhesus monkey or rabbit in the sample.

In a preferred embodiment, the multiplex polymerase chain reaction is performed in a single reaction chamber. In a more preferred embodiment, the plurality of mammalian nucleic acid fragments is selected from the group consisting of RNA, cDNA, and genomic DNA; wherein the mammalian nucleic acid fragments may be isolated from human, mouse, rat, cat, dog, bovine, pig, sheep, goat, horse, Chinese hamster, Green monkey, Rhesus monkey, rabbit, or related genera and species according to the above present invention. In another embodiment, this invention is directed to the multiplex polymerase chain reaction which is performed with an isolated nucleic acid.

In a more preferred embodiment, the multiplex polymerase chain reaction is performed directly with a biological sample selected from the group consisting of a mammalian cell culture or a mix of mammalian and bacterial culture.

In another preferred embodiment, the DNA is isolated from a clinical sample. In another preferred embodiment, the primer pairs are selected from the primers listed in TABLE 1.

In another embodiment, this invention is directed to a method of detecting contaminating mammalian cell lines in a sample, the method comprising: (a) designing a plurality of primers [from] in a plurality of subsequences of cytochrome c oxidase I (COI) or cytochrome b, wherein the primers are (i) specific for cytochrome c oxidase I (COI) or cytochrome b subsequences; (ii) have a low frequency of mutations in primer binding sites; (b) performing a multiplex polymerase chain reaction wherein the plurality of primers amplify a plurality of nucleic acid fragments representing the plurality of subsequences of cytochrome c oxidase I (COI) or cytochrome b; and (c) determining that a sample is positive for a particular contamination from a mammalian cell line. In one embodiment, the contaminating cell line is selected from the group consisting of human, mouse, rat, cat, dog, bovine, pig, sheep, goat, horse, Chinese hamster, Green monkey, Rhesus monkey and rabbit.

In another embodiment, this invention is directed to a diagnostic kit useful to identify contaminating mammalian cells or cell line in a sample comprising: (a) a plurality of primers to amplify a plurality of nucleic acid fragments representing a plurality of cytochrome c oxidase I (COI) and/or cytochrome b subsequences; and (b) reagents to perform a multiplex polymerase chain reaction.

In a preferred embodiment, the reagents comprise a DNA polymerase, nucleotides, and buffers.

In a more preferred embodiment, the primers comprise a plurality of DNA molecules comprising nucleotide sequences listed in TABLE 1.

In one embodiment, the invention is directed to a nucleic acid having the nucleotide sequence of any one of SEQ ID Nos that are listed in TABLE 1, a part thereof and variants thereof which, when in single stranded form, specifically hybridize with a target nucleic acid from a contaminating mammalian cell or cell lines DNA as a probe or as a primer.

In one embodiment, the invention is directed to an oligonucleotide having a nucleotidic sequence of any one of SEQ ID Nos that are listed in TABLE 1.

In one embodiment, the invention is directed to a recombinant plasmid comprising a nucleic acid any one of SEQ ID Nos that are listed in TABLE 1.

In one embodiment, the invention is directed to a recombinant host which has been transformed by a recombinant plasmid comprising a nucleic acid of any one of SEQ ID Nos that are listed in TABLE 1. In a preferred embodiment, the recombinant host is Escherichia coli.

In one embodiment, the invention is directed to diagnostic kits for the detection and/or quantification of the nucleic acids of any combination of the contaminating mammalian cells or cell lines described herein comprising any combination of probes defined herein.

In one embodiment, the invention is directed to diagnostic kits for the detection and/or quantification of the nucleic acids of any combination of the contaminating mammalian cells or cell lines described herein, comprising any combination of oligonucleotide probes defined herein.

In one embodiment, the invention is directed to diagnostic kits for the detection and/or quantification of the nucleic acids of any combination of the contaminating mammalian cells or cell lines described herein comprising any combination of primers defined herein.

In one embodiment, the invention is directed to diagnostic kits for the simultaneous detection of nucleic acids of any combination of the contaminating mammalian cells or cell lines described herein comprising any combination of the probes defined herein.

In one embodiment of this invention, the probes may be any materials having base sequence, preferably any one selected from a group consisting of DNA (Deoxyribose Nucleic acid), RNA (Ribose Nucleic Acid), and nucleic acid analogues such as PNA (Peptide Nucleic Acid), LNA (Locked Nucleic Acid) and HNA (Hexitol Nucleic Acid).

In one embodiment of this invention, the support may be any materials to which the probes can be attached, preferably one selected from a group consisting of slide glass, plastic, membrane, semiconductive chip, silicon and gel. The microarray according to the present invention can be manufactured using conventional methods such as pin microarray, ink jet, photolithography or electric array method.

The following examples and annexes are intended to be illustrative of the various methods and compounds of the invention, rather than limiting the scope thereof.

### EXAMPLE 1

Various embodiments of the invention will now be described by way of a number of examples. The examples are presented solely to further describe certain embodiments of the invention, and are not to be construed as limiting the invention in any way.

### Template Preparation

Genomic DNA from different cell lines representing 14 different species; human, mouse, rat, cat, dog, bovine, pig, sheep, goat, horse, Chinese hamster, Green monkey, Rhesus monkey and rabbit was used for the development of the assay. The cell lines used in this study are: CCL-1, CCL-60, CRL-1430, CRL-2032, CRL 1601, CL-101, CCL-81, CCL-2, CCL-57, CRL-1633, CCL-209, CRL-6306, CCL-73 and CCL-39 (ATCC, Manassas, Va.), K562 (Promega, Madison, Wis.). Purified genomic DNA was extracted from 10⁶ cells using the UltraClean Tissue DNA kit (MoBio, Carlsbad, Calif.).

To test the efficiency and the sensitivity of the developed assay, 10³-10⁶ cultured cells were harvested and then incubated for 15 minutes at 37° C. in the presence of 100 μl of lysis buffer containing: 40 mM Tris acetate pH 7.6, 1 mM EDTA, 0.5% Igepal CA-630 (non-ionic detergent). After inactivation of proteinase K for 10 minutes at 95° C., 5 μl of the cell lysate was used as a template for PCR analyses.

### Oligonucleotide Design and PCR

For multiplex detection of the COI and cytochrome b targets, the designed oligonucleotides were used at specific final concentrations in the master mixes as follows. The following oligonucleotide primers: Hs-F, Hs-R, Rn-F, Rn-R were used in PCR mixes at 100 nM final concentration. Oligonucleotides Mm-F, Mm-Rat were used at 140 nM final concentration The oligonucleotides Cf-F, Cf-R, Ch-F, Ch-R, Ec-F, Ec-R, Bt-F, Bt-R, Cg-F, Cg-R, Oc-F, Oc-R, Fc-F, Fc-R were used at 200 nM final concentration while the oligonucleotides Mm-F, Mm-R, Ca-F, Ca-R were used at 400 nM. Oligonucleotides IC-F and IC-R were added at 40 nM. The same concentrations of specific oligonucleotides were maintained in all of the mixes presented in this report. In each case primers were mixed from stock 10 μM oligonucleotide mixes. A complete list of the oligonucleotide sequences is shown in Table 1.

### PCR Amplification

The PCR buffer consists of: 20 mM Tris-Cl at pH 8.4, 50 mM KCl, 2.5 mM MgCl₂, 5 mM dNTPs, 0.5% glycerol, 0.006% NP40/Tween (1:1), 0.5 units Platinum Taq (Invitrogen, Carlsbad, Calif.) and molecular grade water (ATCC) to make a 50 μl total reaction. The thermocycling conditions were as follows: 95 degrees C. for 5 minutes to activate the enzyme and then 30 cycles of 95 degrees C. for 30 seconds and 60 degrees C. for 1 minute followed by a final elongation of 72 degrees C. for 7 minutes and then an indefinite hold at 4 degrees C. PCR products were visualized on 2% or 4% pre-cast gels stained with ethidium bromide.

### Results:

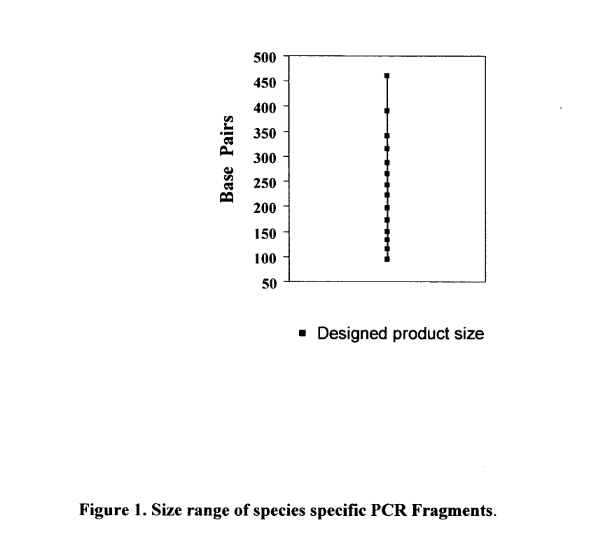

Our objective was to design a sensible assay for species detection and identification. A multiplex PCR-based assay will allow for a rapid detection and identification of cell lines/organism commonly used in research laboratories as well as other industrial areas and thus form the common contaminants found in other systems. In order for a multiplex assay to be successful, the selected oligonucleotides should be able to generate amplicons that are not only specific for each of the detected templates, but are also easily distinguished by the chosen detection method. The oligonucleotides used in this assay are such that the specific amplicons generated by PCR can be separated easily in an agarose gel, a low-cost detection method widely used in any research labs (FIG. 1). The differences in the molecular size of the generated amplicons could also be distinguished as differences in the melting temperature of each of the double-stranded DNA fragments amplified by PCR. As such, this multi-plex assay for species identification could be easily adapted to a closed-tube fluorescence based assay.

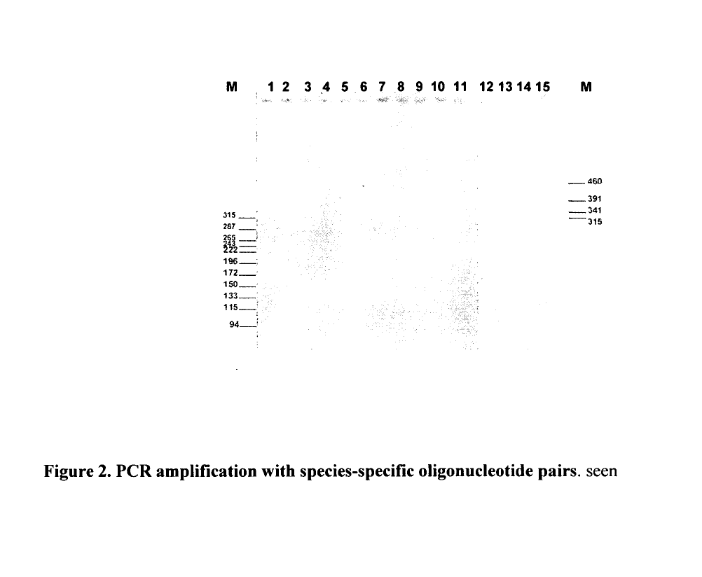

The designed oligonucleotides yielded amplified products that could be distinguished easily by gel electrophoresis (FIG. 2). Thus the size of the amplified products becomes a signature for the presence of a particular species in a particular cell culture. For example, a 150 bp band now becomes an indicator for the presence of mouse in the cell culture. This band is easily distinguishable from its nearest neighbors by size which are rabbit (133 bp) and dog (172 bp). This step-wise pattern maintains for all of the 14 species specific primer pairs and the amplified products they produce and the ease in which they are separated by gel electrophoresis is shown in FIG. 2.

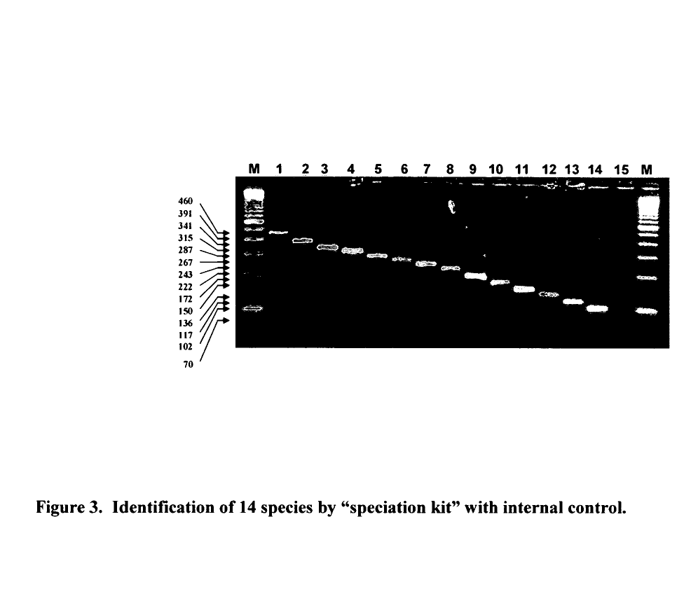

In a PCR assay, the presence of controls is necessary since numerous factors such as inactive polymerase enzyme or improper cycling conditions can render a PCR ineffectual, and therefore a negative result has little diagnostic meaning. The quality of the PCR template and its preparation method are also important for the adequate functioning of the assay. We designed two oligonucleotides specific for the amplification of a conserved region in the 18S rRNA gene. Thus, in the presence of genomic DNA, the generated amplicon will serve as a control for the presence of template and its preparation while a known template can be used as a positive control to monitor the efficiency of the assay. The size of the amplicon generated from the oligonucleotides designed to recognize the 18S rRNA is lower than any of the fragments generated. To ensure that the amplification of the internal control does not interfere with the amplification of the specific targets, the oligonucleotides designed for detection of the internal control was used in a much lower concentration than the other oligonucleotides as described in the Materials and Methods section. FIG. 3 shows the amplification of species specific targets for the 14 species as well as the amplification of the conserved region of the 18S rRNA gene. As shown in the figure each of the specific targets can amplify successfully in the presence of the internal control. The sequences of the 14 designed oligonucleotide pairs and the identity of the generated amplicons is shown in Table 1. Also shown are the sequences of the forward and reverse oligonucleotides for the internal control designed from a highly conserved region of the 18S gene.

The sensitivity of target identification was tested for all the specific targets. Each of the primers was assayed for sensitivity in picking up species specific template DNA as is shown in Table 2. The sensitivity of the assay ranged from 0.01 ng to 0.001 ng of template DNA. Additionally we challenged the sensitivity of the assay in the presence of 1 and 10 ng of non-specific genomic DNA. We observed no reduction in the sensitivity of the assay when background DNA was included (data shown for 1 ng backround).

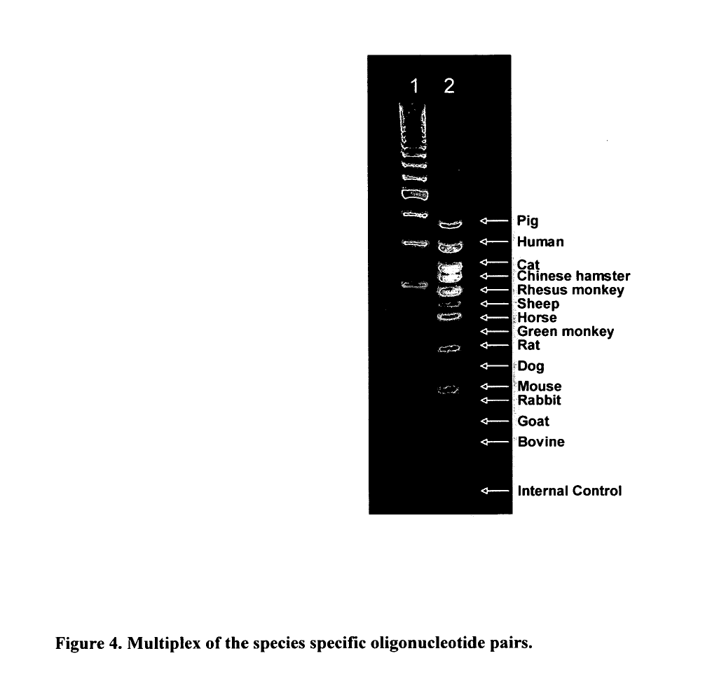

To reduce the time involved in running the assay and to increase simplicity, the detection of specific amplicons was tested in a multiplex assay where each of the species specific primer pairs together with the primers specific for the detection of the internal control, were mixed together as described in the Materials and Methods section. FIG. 4 shows the results of the speciation assay where mixed cell pellets are used as a template. Although specific amplicons for all the 14 species present are detected, detection of fourteen templates in a multiplex assay may become challenging especially if the template used for the analyses is available in low concentration. Moreover, detection of all the fourteen species as presented in the FIG. 4, may not be the focus of each application. To test for the versatility of the assay and to target it for various detection applications we tested the possibility of using the designed oligonucleotides in various PCR mixes where a subset of the templates of interest could be detected.

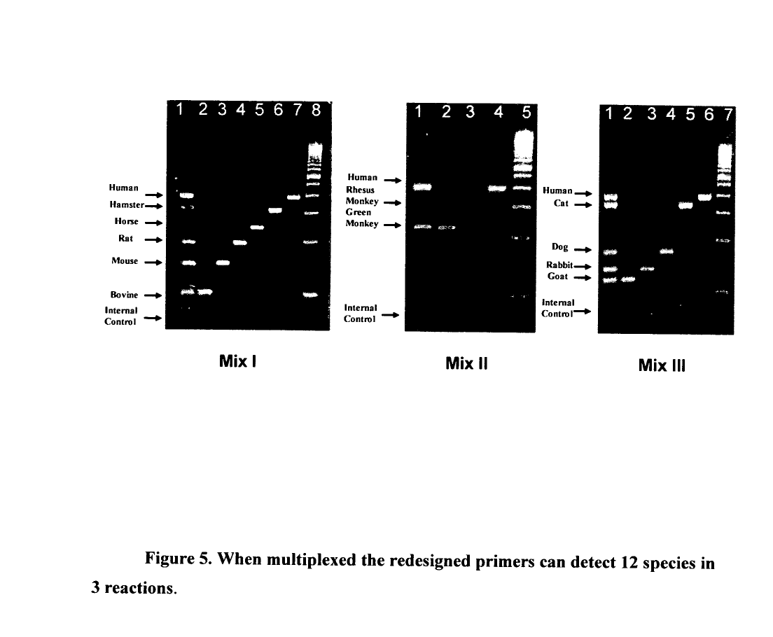

For this, we formulated three different primer mixes that could be used for more specific applications of the assay, 12 of the primer pairs for species specific detection were multiplexed into 3 master mixes (Speciation kit mix I, mixII and mixIII). Each mix contains a subset of the designed primer pairs and the internal control. When used together, the three master mixes can detect the presence or absence of any of the 12 species in the assay and thus show the presence of any interspecies cross-contamination that may have occurred (FIG. 5 A, B, C). Mix I detects 6 species and the internal control (FIG. 5A), mix II detects 3 species and the internal control (FIG. 5B) and mix III detects 5 species and the internal control (FIG. 5C). As a point of reference, oligonucleotides specific for detection of human targets are present in all three mixes. These results show the flexibility of adapting this multiplex method for detection of any subset of species currently used in a particular application. For example, Mix I can detect any cross contamination between human, mouse, rat and bovine cells, commonly used in research laboratories. Our results show that the designed oligonucleotides could be combined in several combinations that allows for the targeting of this assay toward specific applications.

To test the effectiveness of our master mixes in detecting small amounts of interspecies contamination we sought to challenge the mixes using carefully controlled mixtures of mouse and human cell lines. Mouse and human cell lines were selected because they represent extremely common cell lines for laboratory research. The mixed templates were prepared by mixing human cell line (CCL-2) and mouse cell line (CCL-1) as shown in Table 3. In every reaction 1×10⁶ total cells were used as starting material but the cells consisted of different ratios of human and mouse cells. In the most challenging conditions human cells outnumbered mouse cells 99:1 (Table 3, condition I) or conversely mouse cells outnumbered human cells 99:1 (Table 3, condition A). Intermediate conditions with different ratios were also tested as shown in Table 3. Overall, the ratios were designed to mimic cross-contamination of a cell culture at a point prior to overgrowth.

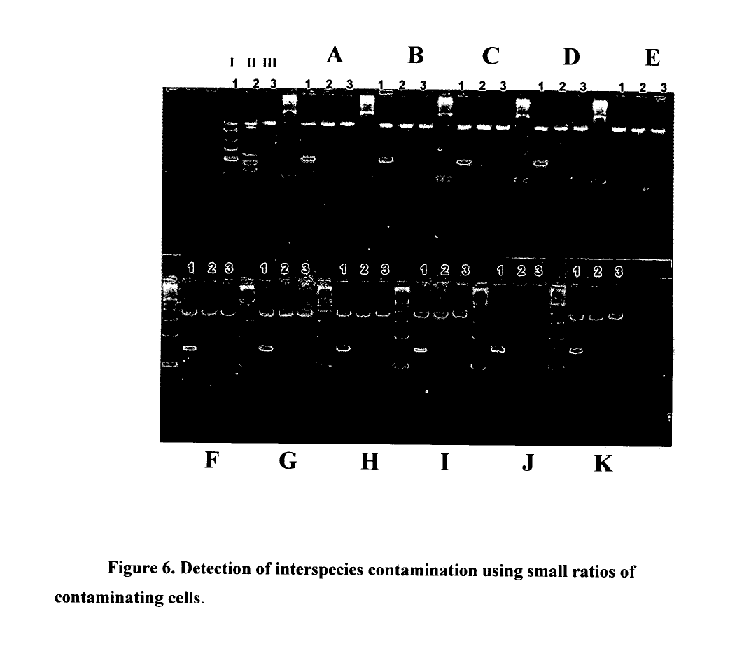

To test for efficiency of amplification and lack of false positives in a multiplex assay, each of the template mixtures shown in Table 2, was tested across each of the three selected master mixes. Each master mix with all template mixtures picked up a positive band for human at 391 base pairs because the human primers are included in each master mix (FIG. 6) with one exception (FIG. 6, panel J) which had only mouse DNA. Thus, the human primer pairs could easily detect the presence of human DNA hidden 1:99 in mouse DNA background (FIG. 6, panel A). Likewise, an amplification product of 150 base pairs indicating the presence of mouse DNA on the selected template was detected using the master mix (I or A) in the all template mixtures tested except for template E (FIG. 6, panel E) which contained only human DNA. The presence of mouse DNA is detected only in master mix I, the only mix that contains oligonucleotides specific for detection of mouse DNA templates. Thus, the mouse primer pairs could easily detect the presence of mouse DNA hidden 1:99 in human DNA background (FIG. 6, panel I). Also, all the amplification products correspond to presence of DNA template in the mixes, unspecific products due to misannealing of oligonucleotides are not observed regardless of high or low amounts of a given template.

### Discussion

A PCR-based method for defining or confirming the species of origin for several animal cell lines and for detecting interspecies cross-contamination has been described previously (10). Here, we have designed oligonucleotides containing sequences specific for Cytochrome oxidase I and Cytochrome b genes that would allow the detection of 14 different species in a multiplex assay and could be easily detected by analyzing the generated amplicons by gel electrophoresis. Thus, the presence of an amplicon of a particular size becomes the signature marker for the presence of one particular species. We also show the possibility of combining the designed primers in several multiplex mixes that could be used for detection of various combinations of the above species. Although all 14 oligo primer pairs with the internal control primers could theoretically be used as a single multiplex such an assay containing 30 primers for 15 different targets might compromise the sensitivity of the assay. Thus we opted to split the primers into smaller multiplexes. These smaller multiplexes allow for the adaptation of this multiplex assay toward a more focused and targeted application.

When the multiplex master mixes were challenged with mixed template from all species they produced signature bands for every species at the same time, all easily detectable by simple size separation on an agarose gel. In a cross-contamination assay a band of amplification was detected in the presence of down to 1% of contaminating cells. The multiplex master mix produced one simple signature band for the 1% contaminating cells and simultaneously one band for the 99% background species. In all cases, regardless of the low ratio of the template or its absence, no unspecific amplicons were observed. These results show that the designed primers do not generate any unspecific products often due to an unspecific annealing of the present oligonucleotides. Also the selection of oligonucleotides, buffer and PCR conditions is such that there is no inhibition of minor templates present in the mix due to favorable amplification of the dominant templates.

The multiplex master mixes can be assembled with lysis buffer, PCR buffer, species specific primers, internal control and positive control DNA (Table 3). In this configuration the species detection ‘kit’ has all of the ingredients necessary for a detection assay ready to use. The procedure is very simple and quick, and the PCR procedure takes less than 3 hours.

Currently the gold standard in species identification is isoenzyme biochemical analysis. Analysis of a cell line with 3-5 isoenzyme substrates can identify or confirm the species of origin of the cell line and can detect and identify interspecies cross-contamination. However the assay requires specific equipment and expensive reagents and very few research laboratories regularly perform the analysis on a routine basis. Furthermore the isoenzyme assay can only detect cross-contamination when the contaminating cell lines comprise 25% (7) or 11% (8) of the total cell population.

PCR by contrast is now a routine in laboratories and requires no dedicated equipment or expensive reagents. As shown, a PCR mix containing primers for a subset of templates can be used quite efficiently providing a great flexibility to this assay and could target it to various applications, such as forensic analyses, detection of contaminations in food samples or detection of intraspecies contaminations in research laboratories.

In research studies 77%-92% of all cell culture systems offered by the main culture collections of the world are from human, mouse, or rat origin (5). Thus, one simple PCR from the multiplex mix I can eliminate most interspecies contamination used for most of cell culture systems used in research laboratories.

### REFERENCES

1. Markovic, O. and N. Markovic (1998). “Cell cross-contamination in cell cultures: the silent and neglected danger.” In Vitro Cell Dev Biol Anim 34(1):
2. Freshney, R. I. (1994). “Culture of Animal Cells.”, John Wiley & Sons, New York: 243-252
3. Denecke, J., K. Becker, et al. (1999). “Falsification of tetrazolium dye (MTT) based cytotoxicity assay results due to mycoplasma contamination of cell cultures”. Anticancer Res 19(2A): 1245-8.
4. Langdon, S. P et al. (2004). “Cell culture contamination: an overview”. Methods Mol Med 88:309-17.
5. O'Brien (2001). “Cell culture forensics”. Proc Natl Acd Sci 14 (98): 7656-7658
6. Stulberg, C. S. (1973). “Extrinsic cell contamination of tissue cultures.” In J. Fogh (Ed.), “Contamination in Tissue culture.” Academic Press, New York: 1-27
7. Nelson-Rees, W. A., D. W. Daniels, et al. (1981). “Cross-contamination of cells in culture.” Science 212(4493): 446-52.
8. Hebert, P. D., A. Cywinska, et al. (2003). “Biological identifications through DNA barcodes.” Proc Biol Sci 270(1512): 313-21.
9. Hebert, P. D., S. Ratnasingham, et al. (2003). “Barcoding animal life: cytochrome c oxidase subunit 1 divergences among closely related species.” Proc Biol Sci 270 Suppl 1: S96-9.
10. Parodi, B., O. Aresu, et al. (2002). “Species identification and confirmation of human and animal cell lines: a PCR-based method.” Biotechniques 32(2): 432-4, 436, 438-40.

FIG. 1. Size range of species specific PCR Fragments. Oligonucleotide sets for fourteen animal and human targets have been designed to produce amplified product that increases in a step-wise fashion for each species. This stepwise design is essential for the development of a multiplexed assay that can be resolved by simple agarose gel electrophoresis. In the figure, each blue square represents the size of the amplified product for a different species. The range is extended from 94 bp to 460 pb and no amplified product is within 20 bp of any other. Thus the size of the amplified product becomes a signature marker for presence of a particular species in a cell culture mix.

FIG. 2. PCR amplification with species-specific oligonucleotide pairs. Amplified products were detected by ethidium bromide staining after agarose gel electrophoresis in a 4% pre-casted gel. The exact size of each product is indicated by the marker column (M1) and is shown in blue. Each lane across the gel shows amplified product specific for a different species for the 14 species in the assay: Lane 1, bovine specific product (94 bp); Lane 2, goat (115 bp); Lane 3, rabbit (133 bp), Lane 4, mouse (150 bp); lane 5, dog (172 bp); Lane 6, rat (196 bp); Lane 7, African green monkey (222 bp); Lane 8, Horse (243 bp); Lane 9, sheep (265 bp); Lane 10, Rhesus monkey (287 bp), Lane 11 Chinese hamster (315 bp); Lane 12 Chinese hamster repeat for reference on the second gel fragment; Lane 13, cat (341 bp); Lane 14, human (391 bp); Lane 15, pig (460 bp); (M2), second marker showing sizes for the second gel fragment. As can be seen the amplified products increase in size from bovine (94 bp) to pig (460 bp) in an easily resolvable step-wise fashion.

FIG. 3. Identification of 14 species by “speciation kit” with internal control. Amplified fragments were detected by ethidium bromide staining on a 2% agarose gel. The label on the far left hand size shows the lengths of the fragments. The internal control can be seen as a band in all PCR reactions at 70 bp and has been multiplexed with each particular primer pair. Note that a few of the primers have been modified slightly from FIG. 2. Lane M, the 100 bp marker (Invitrogen) is present on either side of the gel. Lane 1, pig specific product (460 bp); Lane 2, human (391 bp); Lane 3, cat (341 bp), Lane 4, chinese hamster (315 bp); Lane 5, Rhesus monkey (287 bp); Lane 6, sheep (267 bp); Lane 7, horse (243 bp), Lane 8, African green monkey (222 bp), Lane 9,rat (196 bp), lane 10, dog (172 bp), Lane 11 mouse (150 bp); Lane 12, rabbit (136 bp); Lane 13, goat (117 bp); Lane 14, bovine (102 bp); Lane 15, internal control only (70 bp).

FIG. 4. Multiplex of the species specific oligonucleotide pairs. Amplified fragments were detected by ethidium bromide staining on a 4% Nusieve agarose gel. Lane 1 shows the 100 bp ladder (Invitrogen). Lane 2 shows the multiplex performance of oligonucleotide pairs specific for the following 14 species: pig, human, cat, Chinese hamster, Rhesus monkey, sheep, horse, Green monkey, rat, dog, mouse, rabbit, goat, and bovine. The template for the reactions consisted of 0.5 ng-1.0 ng mixed DNA contributed from all of the species with primers in the master mix.

TABLE 1
Sequences of the oligonucleotides included in the assay/kit.

| | | Forward | | Reverse | |
|---|---|---|---|---|---|
| Size | | primer | | primer | |
| (bp) | Species | Name | Sequence | Name | Sequence |
| 460 | Sus Scrofa (pig) | Ss-F | CT ACT ATC CCT GCC | Ss-R | GAA TAG GAA GAT GAA GCC C |
| | | | AGT T | | (SEQ ID No: 2) |
| | | | (SEQ ID No: 1) | | |
| 391 | Homo sapiens | Hs-F | TAG ACA TCG TAC TAC | Hs-R | TCC AGG TTT ATG GAG GGT |
| | (human) | | ACG ACA CG | | TC |
| | | | (SEQ ID No: 3) | | (SEQ ID No: 4) |
| 341 | Felis catus (cat) | Fc-F | TAT TGC CAT TCC TAC | Fc-R | ACG TTA TAT TGA CTC CTA |
| | | | CGG GGT G | | CAA ACA TAA TC |
| | | | (SEQ ID No: 5) | | (SEQ ID No: 6) |
| 315 | Cricetulus griseus | Cg-F | ACTAACCCGCTTCTTCGC | Cg-R | GCG TAG GCG AAC GGA AGT |
| | (Ch. Hamster) | | ATTC | | ATC |
| | | | (SEQ ID No: 7) | | (SEQ ID No: 8) |
| 287 | Macaca mulatta | Mm-F | CCCACCCAGTTCAACTAA | Mm-R | AATGGTGAAGGATGGGTCG |
| | (Rhesus monkey) | | GC | | (SEQ ID No: 10) |
| | | | (SEQ ID No: 9) | | |
| 267 | Ovis aris (sheep) | Oa-F | CGA TAC ACG GGC TTA | Oa-R | AAA TAC AGC TCC TAT TGA |
| | | | CTT CAC G | | TAA T |
| | | | (SEQ ID No: 11) | | (SEQ ID No: 12) |
| 243 | Equus caballus | Ec-F | CTGCCCTAAGCCTCCTAA | Ec-R | AGAAGTAGGAATGATGGGGG |
| | (horse) | | T | | (SEQ ID No: 14) |
| | | | (SEQ ID No: 13) | | |
| 222 | Cercopithecus | Ca-F | CTTCTTTCCTGCTGCTAA | Ca-R | TTTGATACTGGGATATGGCG |
| | aethiops (Gr. | | TG | | (SEQ ID No: 16) |
| | Monkey) | | (SEQ ID No: 15) | | |
| 196 | Rattus norvegicus | Rn-F | CGGCCACCCAGAAGTGTA | Rn-R | GGCTCGGGTGTCTACATCTAG |
| | (rat) | | CATC | | G |
| | | | (SEQ ID No: 17) | | (SEQ ID No: 18) |
| 172 | Canis familiaris | Cf-F | GAACTAGGTCAGCCCGGT | Cf-R | TTCGGGGGAATGCCATGTC |
| | (dog) | | ACTT | | (SEQ ID No: 20) |
| | | | (SEQ ID No: 19) | | |
| 150 | Mus musculus | Mm-F | ATTACAGCCGTACGCTCC | Mm-R | CCCAAAGAATCAGAACAGATG |
| | (mouse) | | TAT | | C |
| | | | (SEQ ID No: 21) | | (SEQ ID No: 22) |
| 136 | Oryctolagus | Oc-F | CGCC TAT ACA ATA TGA | Oc-R | TGTGG TTG TTA GTT CAA TAG |
| | cuniculus (rabbit) | | AAT ACT GTT | | TCT |
| | | | (SEQ ID No: 23) | | (SEQ ID No: 24) |
| 117 | Capra hircus | Ch-F | ATA TCA ATC GGG TTT | Ch-r | AGT TGG GAT AGC GAT AAT |
| | (goat) | | CTA GGA TTT ATT | | TAT GGT AGT |
| | | | (SEQ ID No: 25) | | (SEQ ID No: 26) |
| 102 | Bos Taurus (cow) | Bt-F | GCTATTCC AAC CGG GGT | Bt-R | GAAAAT AAA GCC TAG GGC |
| | | | AAA AGT C | | TCA C |
| | | | (SEQ ID No: 27) | | (SEQ ID No: 28) |
| 70 | IC | IC-F | CGG GGA ATY AGG GTT | IC-R | GCC TGC TGC CTT CCT TKG |
| | | | CGA TTC | | ATG |
| | | | (SEQ ID No: 29) | | (SEQ ID No: 30) |

The sequences of the 14 oligonucleotide pairs are shown above along with species identification and the size of the amplified product generated by the oligonucleotide pair. The sequence of the internal control designed from an 18S common region is also shown. Bases shown in black are redesigned oligonucleotides for this study. Bases shown in red are from Parodi et al; Species identification and confirmation of human cell lines; A PCR based method. Biotechniques, 32, 2002 432-440.

TABLE 2
Sensitivity of the species specific oligo pairs.

| | | | Genomic DNA |
|---|---|---|---|
| | | | Mixed with 1 ng |
| | | | non-specific |
| | Genomic DNA | Genomic DNA | background DNA |
| Species | (30 cycle PCR) | (35 cycle PCR) | (35 cycle PCR) |
| Bovine | 0.01 ng | 0.001 ng | 0.001 ng |
| Mouse | 0.01 ng | 0.01 ng | 0.01 ng |
| Rat | 0.01 ng | 0.001 ng | 0.001 ng |
| Hamster | 0.001 ng | 0.001 ng | 0.001 ng |
| Horse | 0.01 ng | 0.01 ng | 0.01 ng |
| Human | 0.01 ng | 0.01 ng | 0.01 ng |
| Goat | 0.01 ng | 0.01 ng | 0.01 ng |
| Rabbit | 0.01 ng | 0.01 ng | 0.01 ng |
| Dog | 0.1 ng | 0.001 ng | 0.001 ng |
| Cat | 0.01 ng | 0.001 ng | 0.001 ng |
| Green Monkey | 0.1 ng | 0.01 ng | 0.01 ng |
| Rhesus monkey | 0.1 ng | 0.01 ng | 0.01 ng |
| Sheep | 0.01 ng | 0.001 ng | 0.001 ng |
| Pig | 0.01 ng | 0.001 ng | 0.001 ng |

The sensitivity of the 14 species specific oligo pairs was determined at 30 PCR cycles and at 35 PCR cycles. Shown is the smallest amount of template detectable by the oligonucleotide pairs. The sensitivity was also determined against a constant background of 1 ng of non-specific background DNA. The background DNA consisted of genomic DNA coming from species other than the target species for a given oligo pair.

FIG. 5. When multiplexed the redesigned primers can detect 12 species in 3 reactions. Amplified fragments were detected by ethidium bromide staining on a 4% Nusieve agarose gel. (A) Master mix I detects 6 species; bovine (lane 2), mouse (lane 3), rat (lane 4), horse (lane 5), Chinese hamster (lane 6), human (lane 7). The master mix was challenged against a mixed template of all species and shows positive bands against them all (lane 1). Lane 8 shows the 100 bp ladder (Invitrogen). Note that the internal control is visible at the bottom of every lane. (B) Master mix II detects 3 species; green monkey (lane 2), rhesus monkey (lane 3) and human (lane 4). The master mix was challenged against a mixed template of all of the species and shows positive bands against them all (lane 1). Lane 5 shows the 100 bp ladder. Note that the internal control is visible at the bottom of every lane with mix II. (C) Master mix III detects 5 species; goat (lane 2), rabbit (lane 3), dog (lane 4), cat (lane 5) and human (lane 6). The master mix was challenged against a mixed template of all species and shows positive bands against them all (lane 1). Lane 7 shows the 100 bp ladder. Note that the internal control is visible at the bottom of every lane.

TABLE 3
Ratios of mouse and human cells used as mixed templates.

| Tubes | Cell Nr. | Cell Nr. | Human/Mouse |
|---|---|---|---|
| 1 × 106 Total Cells | CCL-2 (h) | CCL-1 (m) | (%) |
| A | 1 × 10⁴ | 99 × 10⁴ | 1/99 |
| B | 5 × 10⁵ | 99 × 10⁴ | 5/95 |
| C | 1 × 10⁵ | 9 × 10⁵ | 10/90 |
| D | 2 × 10⁵ | 8 × 10⁵ | 20/80 |
| E | 1 × 10⁶ | 0 | 100/0 |
| F | 8 × 10⁵ | 2 × 10⁵ | 80/20 |
| G | 9 × 10⁵ | 1 × 10⁵ | 90/10 |
| H | 95 × 10⁴ | 5 × 10⁵ | 95/5 |
| I | 99 × 10⁴ | 1 × 10⁴ | 99/1 |
| J | 0 | 1 × 10⁶ | 0/100 |
| K | 5 × 10⁵ | 1 × 10⁵ | 50/50 |

In order to test the sensitivity of the mixes at picking up both species in a mixed template mouse (ATCC# CCL-1) and human (ATCC# CCL-2) cell lines were mixed at fixed ratios. Eleven template mixes were prepared (Tubes A-K), all with 1×10⁶ total cells but with varying amounts of human and mouse cells as shown. The final ratio of human to mouse cells is also given. Each of the three multiplex oligonucleotide mixes was challenged with each of the template mixes prepared and the results are shown in FIG. 4.

FIG. 6. Detection of interspecies contamination using small ratios of contaminating cells. Mouse cell line (ATCC# CCL-1) and human cell line (ATCC# CCL-2) were mixed together to produce a variety of fixed ratios (as shown in Table 2). Multiplex mixes I, II, and III containing various combinations of species specific oligonucleotides were then challenged to detect small numbers of “contaminating” cells against a large background. The first three lanes show the amplification of a mixed template where X cell from Y cell lines comprising all 11 species were mixed together and used as a positive control for these specific combinations of oligonucleotides. I; amplification of positive control with master mix I, II; amplification of positive control with master mix II, III; amplification of positive control with master mix III. Panels A-K; amplification of mixed templates as shown in Table 2 (A-K). For all panels PCR results with mix I are shown in the lane 1, mix II in the second lane, and mix III in the third lane. The 100 bp marker (Invitrogen) is shown surrounding each panel. The human and mouse template mixtures vary across panels as follows: (A) Human:mouse 1:99, (B) human:mouse 5:95, (C) human:mouse 10:90, (D) human:mouse 20:80, (E) human:mouse 100:0, (F) human:mouse 80:20, (G) human:mouse 90:10, (H) human:mouse 95:5, (I) human:mouse 99:1, (J) human:mouse 0:100, (K) human:mouse 50:50.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of detecting cellular contamination in a mammalian cell line, the method comprising:
(a) preparing nucleic acid fragments from a sample of cells of a mammalian cell line;
(b) performing a multiplex polymerase chain reaction on the nucleic acid fragments, wherein a plurality of the nucleic acid fragments represent a plurality of mammalian cell lines that are amplified by a plurality of forward and reverse primers; and
(b) determining that the sample is positive for the cellular contamination in a mammalian cell line by detecting amplified nucleic acid fragments in the sample.

2. The method of claim 1, wherein the cellular contamination in a mammalian cell line is selected from the group consisting of human, mouse, rat, cat, dog, bovine, pig, sheep, goat, horse, Chinese hamster, Green monkey, Rhesus monkey and rabbit cellular contamination.

3. The method of claim 1, wherein the plurality of forward and reverse primers is selected from the group consisting of forward and reverse primers whose DNA sequences are selected from the group consisting of cytochrome c oxidase I (COI) and cytochrome b gene coding regions.

4. The method of claim 1, wherein the plurality of forward and reverse primers is selected from the group consisting of primers whose DNA sequences are listed in TABLE 1.

5. The method of claim 1, wherein the plurality of forward and reverse primers comprise the following characteristics:
(a) a length of about three to about 100 bases;
(b) a melting temperature of about 60 degrees C.;
(c) a GC content of about fifty percent;
(d) with minimal dimer formation; and
(e) a primer binding site having low frequency of mutations.

6. The method of claim 5, wherein the plurality of forward and reverse primers further comprise the following characteristics:
the plurality of forward and reverse primers are selected from the group consisting of cytochrome c oxidase I (COI) and cytochrome b subsequences.

7. The method of claim 1, wherein the multiplex polymerase chain reaction is performed with an isolated nucleic acid.

8. The method of claim 1, wherein the multiplex polymerase chain reaction is performed directly with a biological sample selected from the group consisting of mammalian cell culture and bacterial cell culture.

10. The method of claim 9, wherein the bacterial cell culture is isolated from a clinical sample.

10. The method of claim 1, wherein the plurality of primers are selected from primers listed in TABLE 1.

11. A method for detecting cross contamination in a sample, the method comprising:
(a) performing a multiplex polymerase chain reaction amplification on the sample, wherein a plurality of primers amplify a plurality of mammalian nucleic acid fragments selected from the group consisting of nucleic acid molecules of human, mouse, rat, cat, dog, bovine, pig, sheep, goat, horse, Chinese hamster, Green monkey, Rhesus monkey and rabbit according to the above present invention.
(b) determining that the sample is positive for human, mouse, rat, cat, dog, bovine, pig, sheep, goat, horse, Chinese hamster, Green monkey, Rhesus monkey or rabbit mammalian nucleic acid fragments, indicating that a number of amplified fragments is sufficient to detect the human, mouse, rat, cat, dog, bovine, pig, sheep, goat, horse, Chinese hamster, Green monkey, Rhesus monkey or rabbit cross contamination in the sample.

12. The method of claim 11, wherein the plurality of mammalian nucleic acid fragments comprise a plurality of internal fragments.

13. The method of claim 11, wherein the multiplex polymerase chain reaction is performed in a single reaction chamber.

14. The method of claim 11, wherein the plurality of mammalian nucleic acid fragments is selected from the group consisting of RNA, cDNA, and genomic DNA; wherein the mammalian nucleic acid fragments may be isolated from human, mouse, rat, cat, dog, bovine, pig, sheep, goat, horse, Chinese hamster, Green monkey, Rhesus monkey, rabbit, or related genera and species according to the above present invention.

15. The method of claim 11, wherein the multiplex polymerase chain reaction is performed with an isolated nucleic acid.

16. The method of claim 11, wherein the multiplex polymerase chain reaction is performed directly with a biological sample selected from the group consisting of mammalian cell culture and bacterial cell culture.

17. The method of claim 16, wherein the bacterial cell culture is isolated from a clinical sample.

18. The method of claim 11, wherein the plurality of primers are selected from primers listed in TABLE 1.

19. A method of detecting contamination in mammalian cell lines in a sample, the method comprising:
(a) designing a plurality of primers, wherein the plurality of primers are designed from a group consisting of a plurality subsequences of cytochrome c oxidase I (COI) and cytochrome b gene coding regions, wherein the primers are;
(i) specific for either cytochrome c oxidase I (COI) or cytochrome b subsequences;
(ii) have a low frequency of mutations in primer binding sites;
(b) performing a multiplex polymerase chain reaction wherein the plurality of primers amplify a plurality of nucleic acid fragments representing the plurality of subsequences of cytochrome c oxidase I (COI) or cytochrome b gene coding regions; and
(c) determining that the sample is positive for a particular contamination in a mammalian cell line comprising a mammalian cell line selected from the group consisting of human, mouse, rat, cat, dog, bovine, pig, sheep, goat, horse, Chinese hamster, Green monkey, Rhesus monkey and rabbit.

20. A diagnostic kit to identify contamination in a mammalian cell line in a sample comprising:
(a) a plurality of primers to amplify a plurality of nucleic acid fragments selected from the group consisting of a plurality of cytochrome c oxidase I (COI) and cytochrome b subsequences; and
(b) reagents to perform a multiplex polymerase chain reaction.

21. The diagnostic kit of claim 20, wherein the reagents comprise a DNA polymerase, nucleotides, and buffers.

22. The diagnostic kit of claim 20, wherein the primers comprise a plurality of DNA molecules comprising nucleotide sequences listed in TABLE 1.

23. The diagnostic kit of claim 20, wherein the reagents comprise:
(a) a buffer comprising 10 mM Tris-HCl (pH 8.3), 50 mM KCl, 1.6 mM MgCl2, 0.001% (weight/volume) gelatin; and
(b) 0.3 mM dNTP's.

24. The diagnostic kit of claim 20, wherein the plurality of primers have a concentration of about 0.05 μM.

25. The diagnostic kit of claim 24, wherein the concentration of each primer may be about 0.05-0.1 pmol/μl.

26. The diagnostic kit of claim 24, wherein the concentration of each primer may be about 0.1-1.0 pmol/μl.

27. A primer from one of cytochrome c oxidase I (COI) or cytochrome b subsequences wherein the primer is selected from the group consisting of nucleic acid molecules with sequences that are listed in Table 1.
